(19) United States
(12) Patent Application Publication
(10) Pub. No.: US 2009/0043176 A1
(43) Pub. Date: Feb. 12, 2009

(54) HEAT INDICATOR MEASURING DEVICE AND METHOD OF CONTROLLING THE SAME

(75) Inventors: Hideaki Nakajima, Nagano (JP); Masazumi Chino, Nagano (JP)

Correspondence Address: CANTOR COLBURN, LLP, 20 Church Street, 22nd Floor, Hartford, CT, 06103, US

(73) Assignees: CHINON CORPORATION, Nagano (JP); GENESIS TECHNOLOGY CO., LTD, Nagano (JP)

(21) Appl. No.: 12/183,873

(22) Filed: Jul. 31, 2008

(30) Foreign Application Priority Data
Jul. 31, 2007 (JP) 2007-199703
Jun. 25, 2008 (JP) 2008-165365

(51) Int. Cl. A61B 5/00

(52) U.S. Cl. 600/301

(57) ABSTRACT

The invention is to provide a heat indicator measuring device that is able to appropriately prevent a heat disorder. The heat indicator measuring device includes: a temperature measuring unit 2 that measures a temperature, a humidity measuring unit 3 that measures humidity; a heat index calculation unit 5 that calculates a heat index from the measured temperature and humidity; a user data inputting unit 6B that inputs user data as data regarding a user; a noticed content memorizing unit 20 that memorizes a noticed content including a content to be noticed and indicating preventive measures against a heat disorder corresponding to the heat index and the user data; a noticed content determining unit 21 that determines the noticed content based on the heat index and the user data; noticing units 8 and 9 that notice the determined noticed content; and a supporting unit 11 that supports each of the units.

This application is based on Japanese Patent Application No. 2007-199703 filed on Jul. 31, 2007 and Japanese Patent Application No. 2008-165365 filed on Jun. 25, 2008, the contents of which are hereby incorporated by reference.

### BACKGROUND OF THE INVENTION

1. Technical Field

The present invention relates to a heat indicator measuring device and a method of controlling the same.

2. Related Art

A person has a risk of suffering from a heat disorder when he/she does an exercise such as playing a sport, mowing grass and the like under a high temperature and/or high humidity atmosphere. In order to avoid such risk, the patent documents 1 to 4 disclose devices that are provided with a sensor for measuring an atmospheric temperature and relative humidity, determines whether the measured temperature and relative humidity bring a risk of a heat disorder to a person nor not and warns the person such risk if the device determines that there is such risk. In particular, the patent document 4 discloses a device which is wearable for a person with his/her wrist and the like

Further, the patent document 5 discloses a system of noticing a person about a measure, which should be taken by the person, such as supplying water and/or halting his/her exercises in accordance with levels of the risk of a heat disorder in order to prevent a heat disorder. In this system, a monitoring center receives biological data such as a body temperature and the like of the person and a value of wet-bulb globe temperature (WBGT) measured by a WBGT measuring device which is set a place close to the person, among plural WBGT measuring devices set at predetermined places in the country, and then determines whether the person has the risk of suffering from heat disorder or not based on these data

The patent document 6 discloses a structure of a wrist watch provided with a thermal sensor and the like. The patent document 7 discloses a structure of measuring an amount of sweating from a human body and controlling an air conditioning device based on the measured result.

Examples of related arts are the followings: the patent document 1; Japanese patent 376296, the patent document 2; Japanese unexamined patent publication 2003-344175, the patent document 3; Japanese unexamined patent publication 2003-50285, the patent document 4; Japanese unexamined patent publication 2006-345826, the patent document 5; Japanese unexamined patent publication 2005-334021, the patent document 6; Japanese utility model patent 2508710 and the patent document 7; Japanese unexamined patent publication 2003-83590.

According to devices disclosed in the patent documents 1 to 3, however, a person using these devices have to make his/her own decision about what measure should be taken by him/her though these devices can notice him/her about a state of the temperature and relative humidity causing a heat disorder. Hence, such person's own decision probably causes the person not to take an appropriate measure even if he/she acknowledge the risk of a heat disorder.

Further, the patent document 4 has a disadvantage in that a device can not notice a person about an appropriate measure in the case when there is a large difference between the measured WBGT value and the actual WBGT value around the person if a site for measuring environmental temperature is far from a place where the person is actually doing exercises. For example, if a person stays in a boiler room, there is possibility of a large difference between the measured WBGT value and the actual WBGT value around the person, making it impossible to send the person an appropriate notice that corresponds to the environment where the person stays.

### SUMMARY

An advantage of the present invention is to provide a heat indicator measuring device and a method of controlling the same which can appropriately prevent a heat disorder.

According to a first aspect of the invention, a heat indicator measuring device includes: a temperature measuring unit that measures a temperature; a humidity measuring unit that measures humidity; a heat indicator calculation unit that calculates a heat indicator from the measured temperature and humidity; a user data inputting unit that inputs user data as data regarding a user; a noticed content memorizing unit that memorizes a noticed content (including a content to be noticed) showing a preventive measure against a heat disorder corresponding to the heat indicator and the user data; a noticed content determining unit that determines the noticed content, based on the heat indicator and the user data; an noticing unit that notices the determined noticed content; and a supporting unit that supports each of the units.

The first aspect of the invention enables the heat indicator measuring device to easily be placed at a place where a biological object exists and appropriately prevent a heat disorder in response to an environment in which a user stays. Further, the noticing unit is controlled by a program, making it possible to reduce a volume of the memorizing unit and simplify the structure of the heat indicator measuring device.

In addition to the first aspect of the invention, the noticed content may include an instruction message, a sound pattern sounded by a buzzer along with the instruction message, timing of displaying the instruction message and the sounding from the buzzer.

This additional first aspect brings an advantage in that a preventive measure for preventing a heat disorder can be specifically noticed with appropriate timing.

In addition to the first aspect of the invention, the noticed content memorizing unit may be exchangeable.

This additional first aspect enables the heat indicator measuring device to instruct an appropriate measure in response to various conditions.

In addition to the first aspect of the invention, the heat indicator calculation unit may calculate an average of temperatures measured during a predetermined period or by a predetermined number and may use the averaged temperature for calculating the heat indicator.

This additional first aspect prevents the heat indicator calculation unit in the heat indicator measuring device from calculating the heat indicator incorrectly due to the measured temperature which is suddenly raised by the instantaneous irradiation of sun light onto the temperature measuring unit for example. Namely, this additional first aspect improves the accuracy of calculating the heat indicator.

In addition to the first aspect of the invention, the heat indicator calculation unit may calculate an average of humidity measured during a predetermined period or by a predetermined number and may use the averaged humidity for calculating the heat indicator.

This additional first aspect can prevent the heat indicator calculation unit from calculating the heat indicator incorrectly due to the measured humidity which is suddenly raised by applying steam onto the temperature measuring unit for example. Namely, the additional first aspect improves the accuracy of calculating the heat indicator.

In addition to the first aspect of the invention, the biological object data measuring unit may be provided so as to measure biological object data as measured biological object data. The noticed content memorizing unit may memorize a noticed content corresponding to the measured biological object data in addition to the heat indicator and the biological object data. The noticed content determining unit may determine the noticed content, based on the measured biological object data, the heat indicator and the biological object data.

The additional first aspect enables the heat indicator measuring device to notice the biological object and its neighbor about an appropriate noticed content in which the biological object data are considered.

In addition to the first aspect of the invention, a wearing unit which is wearable for a biological body may be provided and detecting portions of temperature and humidity measuring units may be placed at a position opposing to a side of the biological body when the device is mounted on the biological body by the wearing unit.

The additional first aspect can improve the accuracy of measuring an atmospheric temperature and relative humidity measured by the heat indicator measuring device since the effect of body temperature and sweat from the biological object onto the device is reduced when measuring an atmospheric temperature and relative humidity.

In addition to the first aspect of the invention, a biological sensor may be provided on a side opposing to the biological body on which the heat indicator measuring device is mounted.

The additional first aspect enables the heat indicator measuring device to accurately detect biological data of the biological object.

a temperature measuring unit that measures a temperature; a humidity measuring unit that measures humidity; a heat indicator calculation unit that calculates a heat indicator from the measured temperature and humidity; a user data inputting unit that inputs user data as data regarding a user; a noticing unit that notices the user about a preventive measure against a heat disorder; a memory unit that memorizes a program for controlling the noticing unit to notice the preventive measure based on the heat indicator and the user data; a controller controlling the noticing unit for controlling the noticing unit based on the program; and a supporting unit that supports each of the units.

The second aspect of the invention enables a user to easily allocate the heat indicator measuring device to a place where a biological object exists, preventing a heat disorder appropriately in response to an environment in which the user stays.

In addition to the second aspect of the invention, the program may make the noticing unit to notice one of or both of supplying water and taking a rest as the preventive measure against a heat disorder.

This additional second aspect can effectively prevent a heat disorder.

In addition to the second aspect of the invention, the program may control the noticing unit to notice one of or both of supplying water and taking a rest every each of predetermined intervals.

This additional second aspect can effectively prevent a heat disorder.

In addition to the second aspect of the invention, a heat indicator change determining unit may be provided so as to determine the change of the heat indicator along with elapse of time and a state of the change, and the program may control the noticing unit based on the heat indicator after the determination of a predetermined change and the user data if the heat indicator change determining unit determines the predetermined change of the heat indicator.

This additional second aspect of the invention can appropriately prevent a heat disorder in response to the heat indicator after the change even if the heat indicator is changed.

In addition to the second aspect of the invention, in a case when the heat indicator change determining unit determines the predetermined change of the heat indicator, the program may control the noticing unit to notice the initial notice after the determination at the time elapsed by a predetermined period from the time of previous noticing before the predetermined change is determined.

This additional second aspect enables the program to appropriately set timing of the initial noticing after the determination in the case when the change of the heat indicator is determined so as to effectively prevent a heat disorder.

In addition to the second aspect of the invention, the predetermined period is the same of the predetermined interval.

This additional second aspect of the invention enables the program to set timing of the noticing every the predetermined interval even when the change of the heat indicator is determined, making the timing of the noticing further effective so as to prevent a heat disorder.

According to a third aspect of the invention, a method of controlling a heat indicator measuring device includes: measuring an atmospheric temperature; measuring humidity; calculating a heat indicator from the measured temperature and humidity; obtaining user data as data regarding a user; and controlling a noticing unit to notice a preventive measure against a heat disorder based on the heat indicator and the user data.

This third aspect of the invention can prevent a heat disorder appropriately in response to the environment where a user stays.

### BRIEF DESCRIPTION OF THE DRAWINGS

The invention will be described with reference to the accompanying drawings, wherein like numbers reference like elements.

FIG. 1 is a circuit block diagram showing the electrical circuit construction of a heat indicator measuring device in an embodiment of the invention.

FIGS. 2(A) to 2(D) are perspective views showing the appearance of the heat indicator measuring device. FIG. 2(A) is a plain view of the heat indicator measuring device. FIG. 2(B) is a bottom view of the heat indicator measuring device. FIG. 2(C) is a left side view of the heat indicator measuring device. FIG. 2(D) is a front view of the heat indicator measuring device.

FIG. 3 is a table showing details of noticed content data corresponding to a user having a physical strength.

FIG. 4 is a table showing details of noticed content data corresponding to a user having a physical weakness.

FIG. 5 is a flow chart showing operations of the heat indicator measuring device.

FIGS. 6 (A) to 6(J) show details of displaying on a liquid crystal display along each of steps in the flow chart in FIG. 5.

FIGS. 7(A) to (C) are diagrams showing kinds of display of a display 29 in FIG. 6(A).

FIGS. 8(A) to 8(E) are diagrams showing details of displaying on a liquid crystal display in a mode of noticing a preventive measure.

FIG. 9 is a diagram showing a content of displaying on the liquid crystal display of the heat indicator measuring device in FIG. 1 during a period until the time when the content in FIG. 8 (B) is displayed.

FIG. 10 is a flow chart showing a flow for controlling an overall operation of a liquid crystal display and a piezoelectric buzzer.

FIG. 11 is a flow chart showing a flow for controlling concrete operations of a liquid crystal display and a piezoelectric buzzer.

FIG. 12 is a flow chart showing a flow for controlling concrete operations of a liquid crystal display and a piezoelectric buzzer.

### DESCRIPTION OF EXEMPLARY EMBODIMENT

### First Exemplary Embodiment

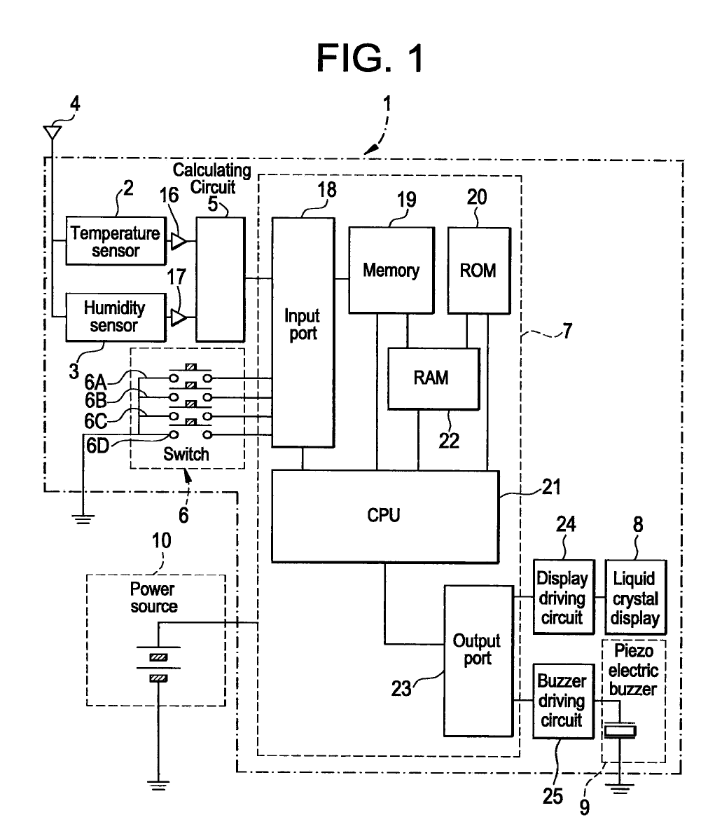

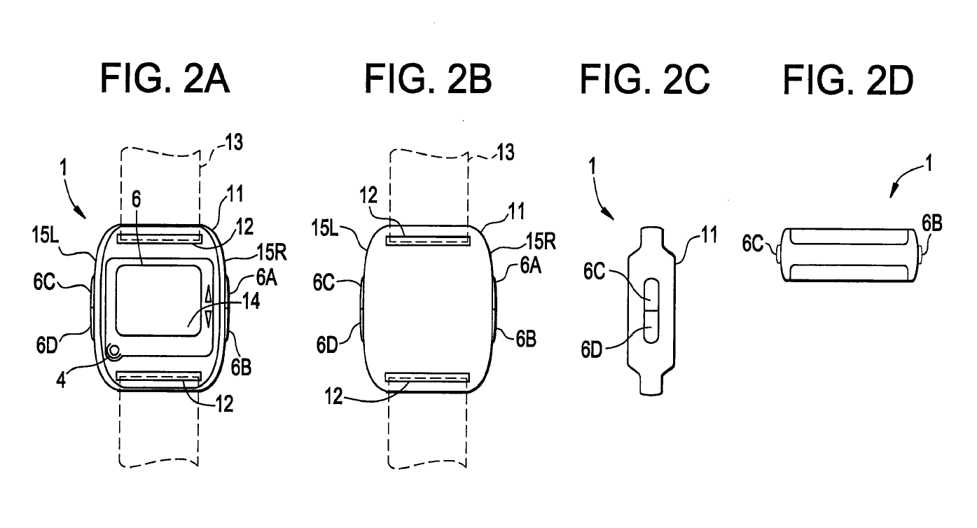

A heat indicator measuring device 1 of a first exemplary embodiment of the invention works as noticing a preventive measure against a heat disorder for a biological object in response to a heat indicator while it measures a heat indicator such as a heat index and a WBGT value. In the exemplary embodiment, using the heat indicator measuring device 1 for a human being (a user) as a biological object will be explained. Hence, the size and the configuration of the heat indicator measuring device 1 are designed so that a user (a person who does an exercise) can do an exercise with wearing it like a watch for example. Such design can give a user an opportunity of doing various exercises such as playing various sports and mowing grass while wearing the heat indicator measuring device 1 with his/her wrist watch, causing a user to easily recognize a preventive measure against a heat disorder in response to the heat indicator of the environment where he/she is placed. Specific configurations of the heat measuring device 1 will be explained with referring to FIGS. 1 and 2.

FIG. 1 is a circuit block diagram showing the electrical circuit configuration of a heat indicator measuring device 1. FIGS. 2(A) to 2(D) are perspective views showing the appearance of the heat indicator measuring device 1. FIG. 2(A) is a plain view of the heat indicator measuring device 1. FIG. 2 (B) is a bottom view of the heat indicator measuring device 1. FIG. 2(C) is a left side view of the heat indicator measuring device 1. FIG. 2(D) is a front view of the heat indicator measuring device 1.

As shown in FIG. 1, the heat indicator measuring device 1 comprises a temperature sensor 2, a humidity sensor 3, a detector 4, a calculating circuit 5, a switch portion 6 a control IC 7, a liquid crystal display 8, a piezoelectric buzzer 9 and a power source 10. The temperature sensor 2 measures an atmospheric temperature. The humidity sensor 3 measures humidity. The detector 4 detects a temperature and humidity measured by the temperature sensor 2 and humidity sensor 3. The calculating circuit 5 calculates a heat indicator and a WBGT value based on the outputs from the temperature sensor 2 and humidity sensor 3. The switch portion 6 inputs various instructions and information to the heat indicator measuring device 1. The control IC 7 controls various operations of the heat indicator measuring device 1. The liquid crystal display 8 displays a notice given to a user. The piezoelectric buzzer 9 sounds various sounds as notices. The power source 10 is composed of a lithium battery and the like.

Further, as shown in FIGS. 2(A) to 2(D), the heat indicator measuring device 1 includes a container 11 as a supporting unit holding constituents such as the temperature sensor 2, the humidity sensor 3 and the like for the heat indicator measuring device 1. The shape of the container 11 is a flat square as a whole and a band hooking up portion 12 is installed at both ends of the container 11 toward the longer direction. The heat indicator measuring device 1 is wearable for a user with his/her wrist by a band 13 as a wearing unit being hooked to the band hooking up portion 12. The container 11 is designed as a size of 4 cm as a longitudinal direction×3 cm as a lateral direction×1 cm as a thickness for example in order that the size does not interrupt user's exercise when a user wears it with his/her wrist.

A display 14 (shown in FIG. 2(A)) of the liquid crystal display 8, the switch portion 6 and the detector 4 of the temperature sensor 2 and the humidity sensor 3 among components shown in FIG. 1 are assembled in the container 11 so that they faces the outer periphery of the container 11. Namely, the display 14 of the liquid crystal display 8 is placed on an upper surface as one of a pair of wider-width surfaces being toward the flat direction of the container 11. Further, the detector 4 is placed on the upper surface of the container 11 and between the circumference of the display 14 and the outer periphery of the container 11. Further, each two of four switches 6A, 6B, 6C and 6C constituting the switching portion 6 are placed on each of a pair of side surfaces 15R and 15L. Each of the pair of side surfaces 15R and 15L is a side surface along the flat direction of the container 11 and the longitudinal direction of it. Namely, switches 6A and 6B are placed on the side surface 15R and switches 6C and 6D are placed on the side surface 15L.

The temperature sensor 2 is a band gap type temperature sensor for example. The humidity sensor 3 is an electrostatic capacitance type relative humidity sensor for example so as to detect relative humidity. In the exemplary embodiment, the temperature sensor 2 and the humidity sensor 3 are integrated into one chip and the monotonic detector 4 measures both temperature and relative humidity of an air with which the detector 4 contacts. A user wears the heat indicator measuring device 1 with his/her wrist so that the display 14 is toward the upper direction (opposing to the skin of the user), making the user look at the display 14 of the liquid crystal display 8. Hence, when the heat indicator measuring device 1 is mounted on a user's wrist, the detector 4 is placed on a front surface, an upper side opposing to a human body with respect to the heat indicator measuring device 1. Namely, the detector 4 is placed at a position where the detector rarely receives effects of human body's temperature and sweat from a user. Hence, this placement allows temperature sensor 2 and the humidity sensor 3 to measure atmospheric temperatures and relative humidity under the condition where human body's temperature and sweat from a user rarely affect the measurement.

Signals regarding a temperature and relative humidity output from the temperature sensor 2 and the humidity sensor 3 are amplified by amplifiers 16 and 17 and then output to the calculating circuit 5. The calculating circuit 5 calculates two heat indicators such as a heat index and a value approximated to a WBGT value (called as a WBGT approximated value.) The heat index is a value calculated based on temperatures and humidity by the following formula (1) suggested by the Australian bureau of weight and measurements. The index shows the degree of hotness felt by a human being.

Heat Index=Ta+0.33×E−0.70×ws−4.00 (1)

E=(Rh/100)×6.105×exp{17.27×Ta/(237.7+Ta)}

Where, Ta is a atmospheric temperature indicated as ° C., Rh is relative humidity indicated as %, and ws is a wind speed (m/s, the value at the height 10 m.) Here, in the heat indicator measuring device 1 of the exemplary embodiment, the wind speed is calculated as 0 m/s.

On the other hand, the WBGT approximated value is also calculated based on temperatures and humidity by the following formula (2) suggested by the Australian bureau of weight and measurements.

WBGT approximated value=0.567×Ta+0.393×E+3.94 (2)

E=(Rh/100)×6.105×exp{17.27×Ta/(237.7+Ta)}

Where Ta is an atmospheric temperature indicated as ° C. and Rh is relative humidity indicated as %.

Here, the WBGT value is calculated by the following formula (3) using an atmospheric temperature (a dry-bulb temperature), a wet-bulb temperature and a black-bulb temperature (heat radiation).

WBGT value=0.7×(wet-bulb temperature)+0.2×(black-bulb temperature)+0.1×(dry-bulb temperature) (3)

The WBGT value is an index for determining a degree of a risk along with an exercise under an extreme heat environment. Calculation of the WBGT value needs three measured values such as a dry-bulb temperature, a wet-bulb temperature and a black bulb temperature and a means for measuring them, making the measuring device a large size. Hence, the Australian bureau of weight and measurements proposes the above formula (2) which can easily calculate the WBGT approximated value approximated to the WBGT value using two measured values such as an atmospheric temperature and relative humidity. The WBGT approximated value does not depend on the black-bulb temperature and obtained from measured values such as atmospheric temperature and relative humidity around a user, which are relatively easily measured. Accordingly, obtaining the WBGT approximated value as a heat indicator simplifies a structure of the heat indicator measuring device 1 that includes two sensors such as the temperature sensor 2 and the relative humidity sensor 3 without including the means for measuring the black bulb temperature if the heat indicator measuring device 1 obtains the WBGT approximated value as a heat indicator.

The control IC 7 comprises an input port 18, a memory 19, a read only memory (ROM) 20, a central processing unit (CPU) 21, a random access memory (RAM) 22 and an output port 23 and the like. The input port 18 is connected to the temperature sensor 2 and the humidity sensor 3 through the calculating circuit 5 and switches 6A to 6D. The output port 23 is connected to the liquid crystal display 8 through the display driving circuit 24 and the piezoelectric buzzer 9 through a buzzer driving circuit 25.

Signals from switches 6A to 6D and signals regarding a heat indicator (a heat index and a WBGT approximated value) output from the calculating circuit 5 are input to the input port 18. Information regarding a heat indicator and information input from switches 6A to 6D are memorized by the memory 19.

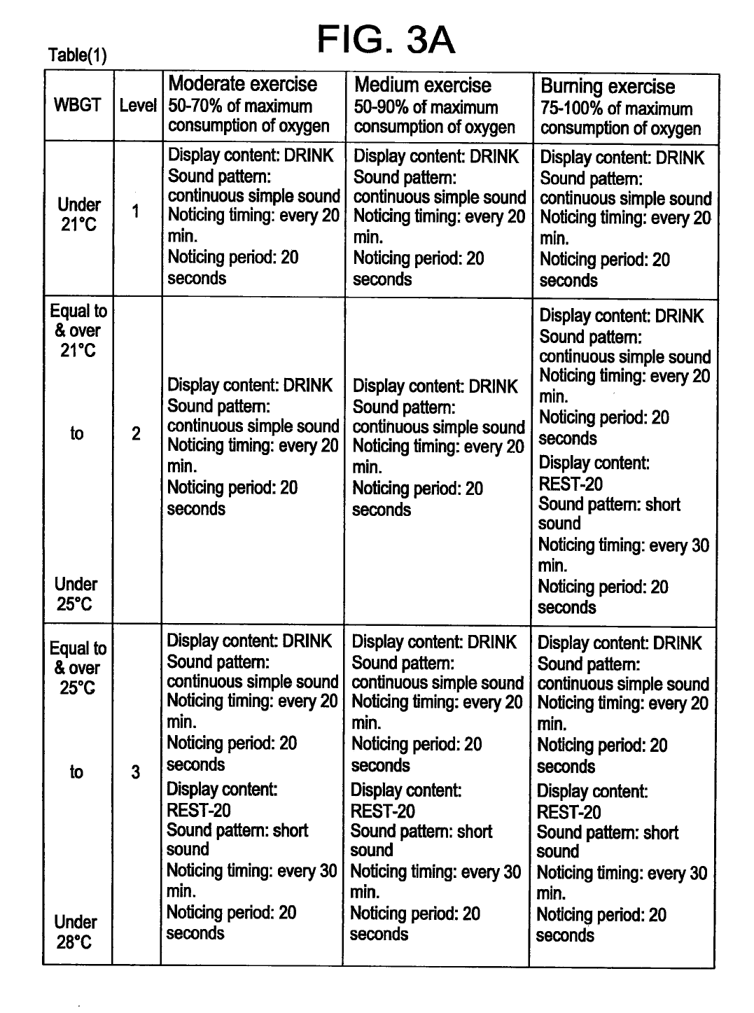

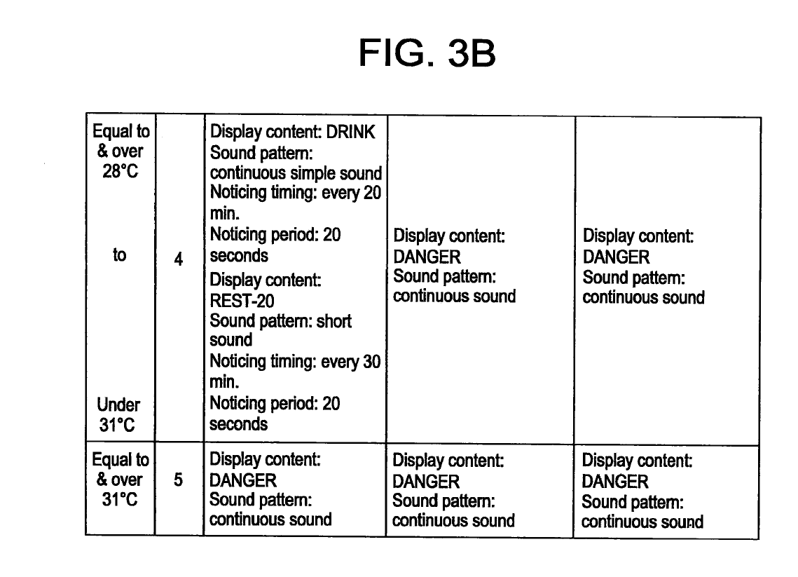

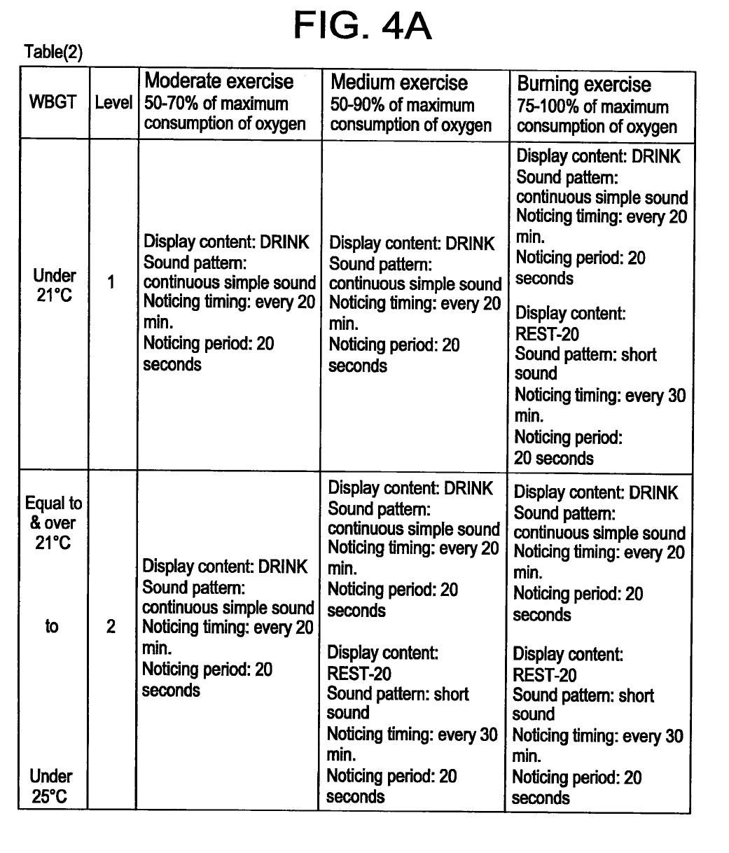

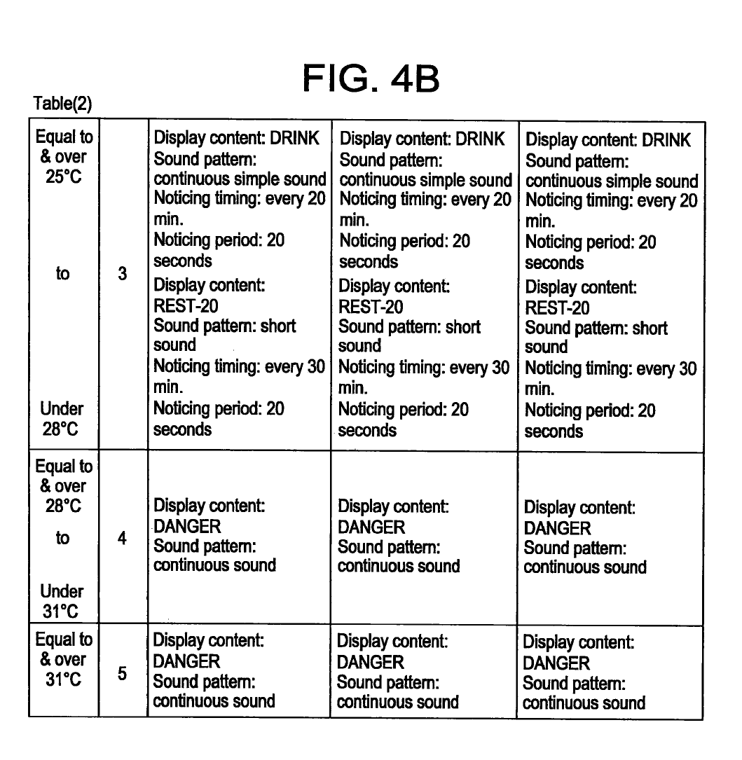

The table (1) shown in FIG. 3 and the table (2) shown in FIG. 4 are memorized as a data table (called as a table of noticed content data, and noticed content data includes content data to be noticed) by ROM 20 which works as memorizing noticed content data. In the table (1) and the table (2), noticed contents (including contents to be noticed) are regulated for noticing a user about WBGT approximated values and preventive measures against a heat disorder corresponding to a level of exercising. The table (1) corresponds to a user having a physical strength and the table (2) corresponds to a user having a physical weakness. In the exemplary embodiment, exercise levels are sorted into three stages, “moderate exercise (the maximum oxygen consumption: 50 to 70%)”, “medium exercise (the maximum oxygen consumption: 50 to 90%)” and “burning exercise (the maximum oxygen consumption: 75 to 100%)”. The WBGT approximated values are sorted into five stages such as the level 1 to level 5 (called as WBGT levels) as the levels of risks of suffering a heat disorder in each of tables.) Namely, in the tables (1) and (2), the noticed contents, which should be noticed to a user as preventive measures against a heat disorder, are regulated so as to correspond to WBGT levels and exercised levels. Hence, specifying any of WBGT approximated values and exercised levels in the tables (1) and (2) can identify a specific preventive measure against a heat disorder corresponding to the specified WBGT approximated value and exercised level.

The heat indicator measuring device 1 notices a user about preventive measures against a heat disorder using the liquid display 8 and the buzzer 9. Hence, noticed contents as preventive measures include an instruction message to a user as being displayed by the liquid crystal display 8, a sound pattern sounded by the piezoelectric buzzer 9, timing of displaying the instruction message and sounding a buzzer sound and the length of a period for displaying the instruction message and sounding a buzzer sound.

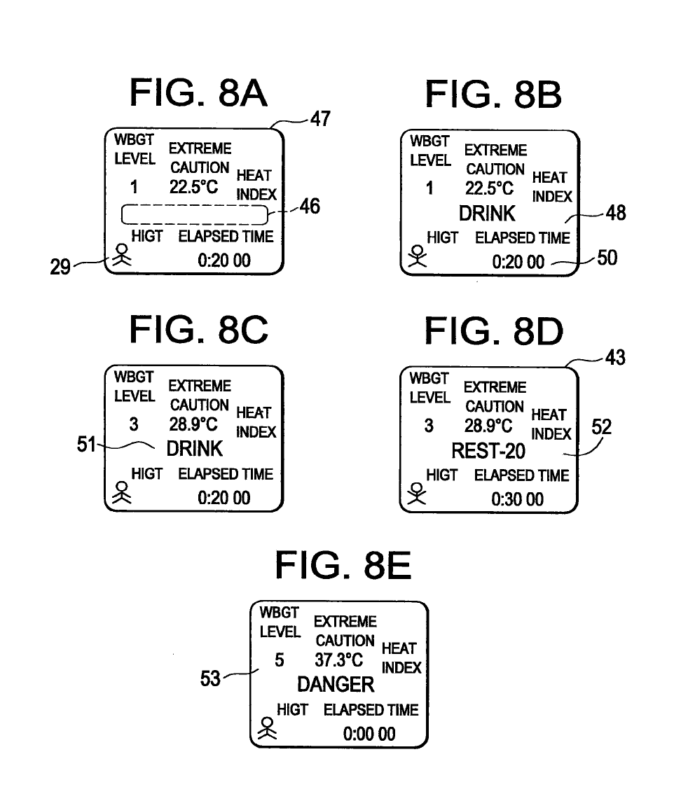

For example, if an exercise level is the moderate level and the WBGT approximated value is “1” in the table (1), the noticed contents are: supplying water (“DRINK” shown in FIG. 8 (B)) displayed by the liquid crystal display 8, a continuous monotonic buzzer sound sounded by the piezoelectric buzzer 9 along with the above display. The display and the sounding are simultaneously performed during the 20 seconds every the timing of 20 minutes.

Further, if an exercise level is the moderate level and the WBGT approximated value is “3” in the table (1), the noticed contents are: taking a rest (“REST-20” shown in FIG. 8 (D)) displayed by the liquid crystal display 8, a short buzzer sound sounded by the piezoelectric buzzer 9 along with the above display, which are preformed during 20 seconds every 30 minutes, in addition to the display of supplying water (“DRINK” shown in FIG. 8 (C)) displayed by the liquid crystal display 8, the continuous monotonic buzzer sound sounded by the piezoelectric buzzer 9 along with the above display, which are performed during 20 seconds every 20 minutes.

Further, if an exercise level is the moderate level and the WBGT approximated value is “5” in the table (1), the noticed contents are: halting an exercise (“DANGER” shown in FIG. 8 (E)) displayed by the liquid crystal display 8, and a continuous buzzer sound sounded by the piezoelectric buzzer 9 along with the above display.

As described above, the table (1) corresponds to a user having a physical strength and the table (2) corresponds to a user having a physical weakness. Namely, it is preferable that a user having a physical weakness should receive a notice of the following preventive measure against a heat disorder. The preventive measure has the same content for a user having a physical strength, but belongs to a low WBGT approximated value compared to that for a user having a physical strength. Hence, in the table (2) of the exemplary embodiment, preventive measures against a heat disorder which are the same contents described in the table (1) are set at the lower WBGT approximated value compared to that in the table (1). More specifically, the preventive measures against a heat disorder taken at the WBGT approximated value “5” in the case of the exercise level “moderate exercise” in the table (1) is a preventive measure taken at the WBGT approximated value “4” in the table (2).

CPU 21 controls various operations of the heat indicator measuring device 1 such as driving the liquid crystal display 8 and the piezoelectric buzzer 9 and the like. Further, CPU 21 works as a noticed content determining unit. Namely, CPU 21 reads out the WBGT approximated value memorized in the memory 19, the information input by switches 6A to 6D and tables of noticed content data (the tables (1) and (2)) memorized in ROM 20 and send them to RAM 22. Then, it determines the noticed content, corresponding to the WBGT approximated value and the information input by switches 6A to 6D, from contents in the tables of noticed content data. Further, CPU 21 allows the liquid crystal display 8 to display a predetermined instruction message and the piezoelectric buzzer 9 to sound a predetermined patterned buzzer sound in response to the determined noticed content.

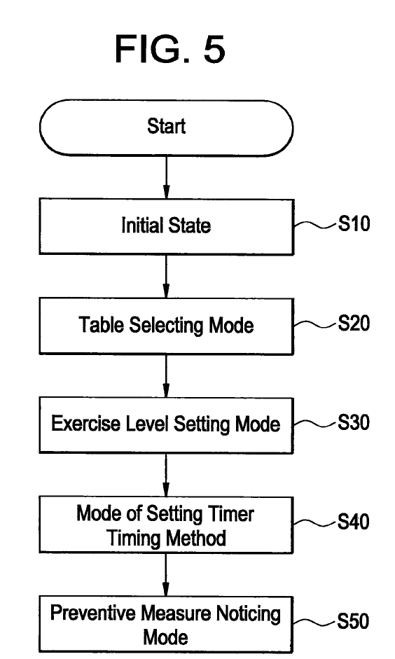

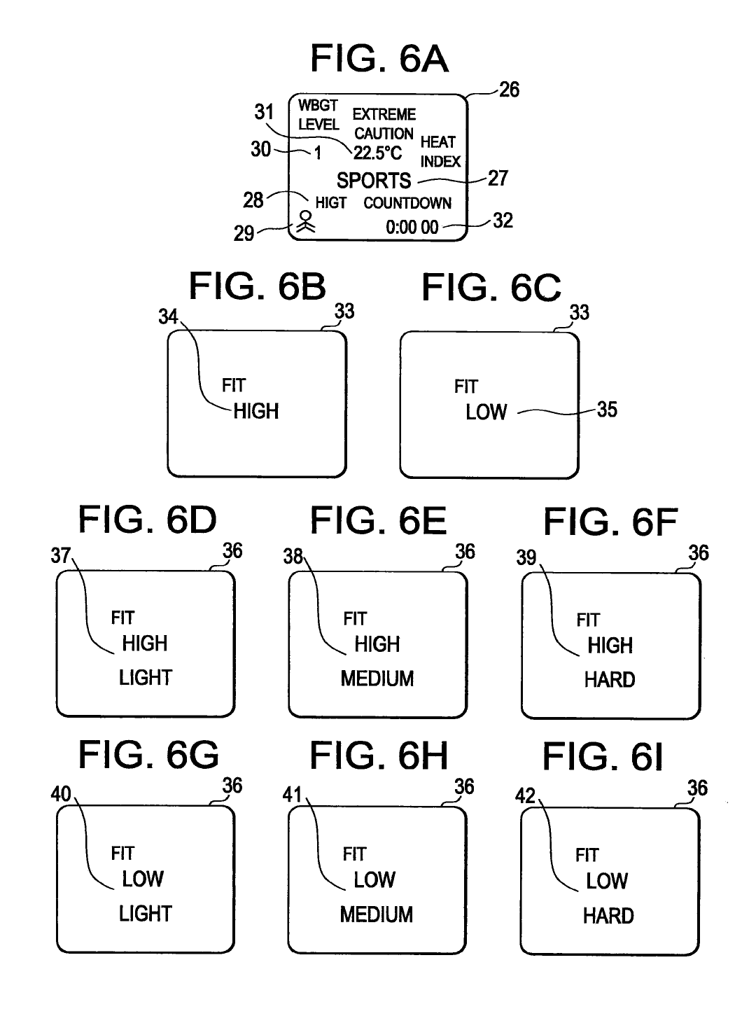

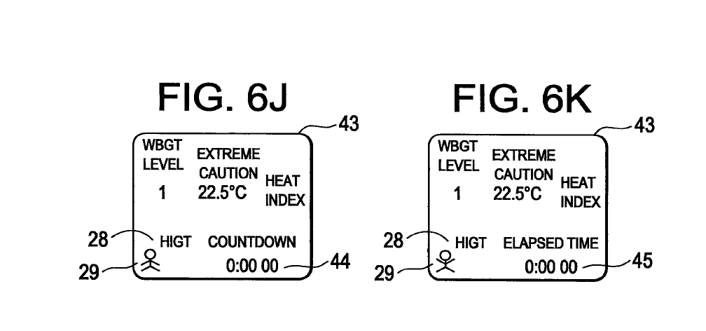

Next, a method of using the heat indicator measuring device 1 will be explained referring to FIGS. 5 and 6(A) to 6(J). FIG. 5 is a flow chart of setting the operation of the heat indicator measuring device 1 and FIGS. 6 (A) to 6(J) are diagrams showing contents in each of steps of the flow chart shown in FIG. 5, displayed by the liquid crystal display 8.

The heat indicator measuring device 1 in the exemplary embodiment works not only as a heat indicator measuring device such as measuring the above heat indicator and noticing a preventive measure against a heat disorder, but as a wrist watch and a stop watch. The switch 6D is to select any one of these three functions. Every time when the switch 6D is pushed, CPU 21 circularly changes the operational mode of the heat indicator measuring device 1 into the heat indicator measuring mode enabling the device to function as a heat indicator measuring device, the wristwatch mode enabling the device to function as a wrist watch, and the timing mode enabling the device to function as a stop watch. Here, it will be explained hereafter a case when the heat indicator measuring mode is selected as the operational mode of the heat indicator measuring device 1 and the heat indicator measuring device 1 functions as a heat indicator measuring device.

If the heat indicator measuring mode is selected by the switch 6D, the heat indicator measuring device 1 becomes an initial state for functioning as a heat indicator measuring device (step S 10.) In the initial state (step S 10), an initial display 26 shown in FIG. 6 (A) is displayed by the liquid crystal display 8. “SPORTS” as a display 27 in the initial display 26 indicates that the mode of the heat indicator measuring device 1 is the heat indicator measuring mode. Further, a display 28 indicates that one of the table (1) and the table (2) is read in RAM 22. If the display 28 shows “HIGH”, the table (1) is read in RAM 22. If the display 28 shows “LOW”, the table (2) is read in RAM 22.

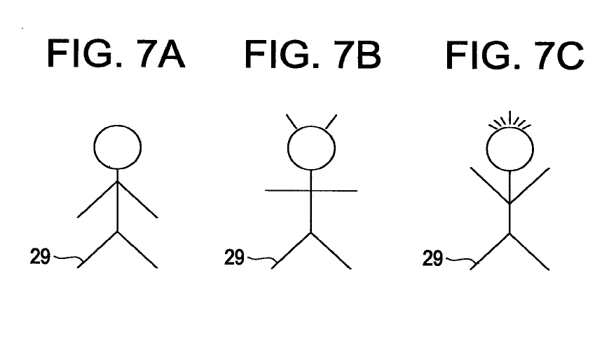

Further, a display 29 indicates that one of preventive measures is specified by designating one of exercise levels when CPU 21 specifies one of preventive measures against a heat disorder based on the tables (1) and (2). If an exercise level is “a moderate exercise”, a display shown in FIG. 7 (A) appears. If an exercise level is “an intermediate exercise”, a display shown in FIG. 7 (B) appears. If an exercise level is “a burning exercise”, a display shown in FIG. 7(C) appears. In the initial state (step S 10), CPU 21 reads the data in the table (1) and send it to RAM 22 as a default operation and designates an exercise level as the “moderate exercise”. Hence, at the time of the initial display 26, “HIGH” appears as the display 28 which is shown in FIG. 6(A) and the content shown in FIG. 7 (A) appears as the display 29.

The heat indicator measuring device 1 starts to measure a WBGT approximated value and a heat index when the heat indicator measuring mode is selected by the switch 6D. Then, the value of the WGBT level in the table (1) corresponding to the measured WBGT approximated value is displayed as a display 30. Namely, CPU 21 determines the WGBT level in the table (1) corresponding to the measured WBGT approximated value based on the measured WBGT approximated value and the table (1) read in RAM 22 as a default. This WGBT level is displayed as the display 30. A user acknowledges the numerical level of a risk of suffering from a heat disorder in the current state by looking at the display 30. Further, the measured heat index is shown as a display 31. A user acknowledges a heat index by looking at the display 31. A display 32 is a timer display described later.

One of operational modes shown in steps S 20, S 30, S 40 and S 50 is moved to the other in series from the initial state (step S 10) every pushing the switch 6A. Then, a predetermined operation described later is set at each of operational modes.

If the switch 6A is pushed one time in the initial state (step S 10), the operational mode becomes a table selecting mode where any one of noticed contents in the table (1) and the table (2) is selected, read and sent to RAM 22 (step S 20.) In the table selecting mode (step S 20), a table selection display 33 shown in FIG. 6 (B) is displayed by the liquid crystal display 8. In the table selecting mode (step S 20), pushing the switch 6B enables to any one of noticed contents in the table (1) and the table (2) be selected, read and sent to RAM 22. In the table selection display 33, a display 34 (FIT/HIGH) shown in FIG. 6(B) and a display 35 (FIT/LOW) shown in FIG. 6(C) are alternatively displayed every time when the switch 6B is pushed. When the display 34 is displayed, the noticed content data in the table (1) is read in RAM 22. When the display 35 is displayed, the noticed content data in the table (2) is read in RAM 22. As described above, the table (1) includes contents of measures for a person having a physical strength and the table (2) includes contents of measures for a person having a physical weakness. Namely, pushing the switch 6B by a user selects one of noticed contents in the tables (1) and (2), inputting user data about the physical strength or weakness of the user into RAM. The switch 6B works as a biological object's (user's) data inputting unit and the user instructs CPU 21 to read one of noticed contents in the tables (1) and (2) corresponding to his/her physical strength and send it to RAM 22 by pushing the switch 6B. Here, the noticed contents in the tables (1) and (2) correspond to a user's physical strength or weakness. Otherwise, noticed content data corresponding to kinds of users such as old persons, children, athletes and the like may be held every one of kinds of users in these tables.

After selecting one of the noticed contents in the tables (1) and (2), pushing the switch 6A one time allows the table selecting mode (step S 20) to be confirmed and the operational mode to be moved to the exercise level setting mode at the same time (step S 30). In the exercise level setting mode (step S 30), an exercise level setting display 36 shown in FIG. 6(D) is displayed by the liquid crystal display 8. In the exercise level setting display 36, a display 37 (“FIT/HGHT/LIGHT”) a display 38 (“FIT/HGHT/MEDIUM”) and a display 39 (“FIT/HGHT/HARD”), which are shown in FIGS. 6(D), 6(E) and 6(F) respectively, are cyclically displayed. If the table (2) is selected as a table of noticed content data in step S 20, a display 40 (“FIT/LOW/LIGHT”) a display 41 (“FIT/LOW/MEDIUM”) and a display 42 (“FIT/LOW/HARD”) which are shown in FIGS. 6(G), 6(H) and 6(I) respectively, are cyclically displayed in the exercise level setting mode (step 30). In the exercise level setting mode (step 30), pushing the switch 6B described above selects one of exercise levels in the noticed contents in the tables (1) and (2) such as the “moderate exercise”, the “medium exercise” and the “burning exercise”. The “moderate exercise” is selected when displays “37” and “40” are displayed. The “medium exercise” is selected when displays “38” and “41” are displayed. The “burning exercise” is selected when displays “39” and “42” are displayed. Namely, pushing the switch 6B enables a user to select one of exercise levels such as the “moderate exercise”, the “medium exercise” and the “burning exercise” and input exercise levels such as an exercise to be exercised by a user or an exercise which is currently exercised by the user as the user data. Namely, the switch 6B works as a biological object's (user's) data inputting unit. The user inputs a level of an exercise, which he/she is to do soon or currently doing, to the heat indicator measuring device 1 by pushing the switch 6B.

Pushing the switch 6A one time after setting the exercise level confirms the selection of the exercise level (step S 30) and allows the operational mode to be moved to a mode of setting a timer timing method where a method of a timer is selected and set for timing (step S 40). In the mode of setting a timer timing method (step S 40), a display 43 of setting a timer for timing shown in FIGS. 6 (J) and 6(K) is displayed by the liquid crystal display 8. Hence, time (or a period) measured by a timer mechanism not shown in the figure is displayed. As a method of measuring time by the timer, one of a subtraction method (counting down) and an addition method (measuring elapsed time) is selected. A display 44 (“COUNTDOWN”) shown in FIG. 6 (J) and a display 45 (“ELAPSED TIME”) shown in FIG. 6(K) are alternatively displayed every pushing the switch 6B. The subtraction method is selected when the display 44 is displayed. The addition method is selected when the display 45 is displayed. Here, in the display 43 of setting a timer for timing, the display 28 corresponding to the selected content in the table selecting mode (step S 20) and the display 29 corresponding to the selected content in the exercise level setting mode (step S 30) are also displayed.

Pushing the switch 6A one time after setting a timer for timing described above confirms the selection of the method of timing by a timer (step S 40) and allows the operational mode of the heat indicator measuring device 1 to be moved to a preventive measure noticing mode (step S50) where any of preventive measures against a heat disorder are noticed based on the operational setting, which is done from steps S 20 to S 40.

Here, the operation of the heat indicator measuring device 1 for noticing preventive measures against a heat disorder will be explained based on the following three premises for example. The table (1) for a user having a physical strength is selected in step S 20. The “moderate exercise” is selected as an exercise level in step S 30. Addition method is selected as the method of timing by a timer in step S 40. In the preventive measure noticing mode (step S50), a display 47 for noticing a preventive measure is displayed as shown in FIG. 8 (A). The display 47 includes a display region 46 showing an instruction message corresponding to the content of the preventive measure.

CPU 21 determines that the measured WBGT approximated value corresponds to any one of WBGT levels in the table (1) and notices the content based on the determined WBGT level in the preventive measuring noticing mode (step S50). For example, if the temperature measured by the temperature sensor 2 is 24° C. and the relative humidity measured by the humidity sensor 3 is 25%, the WBGT approximated value is calculated as around 20° C. by the heat indicator calculation unit 5. Hence, CPU 21 determines that the WBGT level is “1” based on the calculated WBGT approximated value using the table (1). Namely, the exercise level set in step S 30 is the “moderate exercise”. This exercise level allows the noticed content in the table (1) at the time of the WBGT level “1” to become the following. In the content, it takes 20 seconds to display a message of supplying water and sound a continuous monotonic buzzer sound along with the displaying and these displaying and sounding are performed every 20 minutes. Namely, as shown in FIG. 8 (B), CPU 21 instructs the liquid crystal display 8 to display a display 48 (“DRINK”) as a message of supplying water for a user at the location of the displaying region 46 shown in FIG. 8 (A) and repeat it during 20 seconds every 20 minutes, and the piezoelectric buzzer 9 to sound a continuous monotonic buzzer sound during 20 seconds every 20 minutes along with the a period and timing of displaying the display 48.

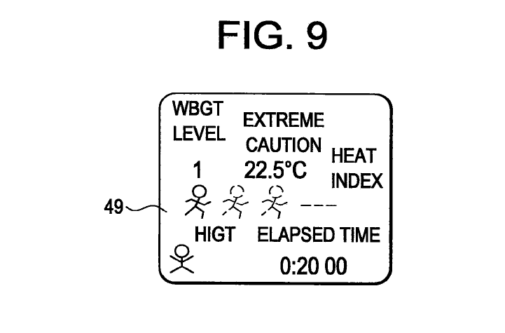

A user acknowledges that supplying water is necessary as the preventive measure against a heat disorder by looking at the display 48 displayed by the liquid crystal display 8. Further, the user recognizes the displaying of an instruction message by hearing the buzzer sound since the piezoelectric buzzer 9 sounds the sound along with the display 48, avoiding missing out the instruction message. CPU 21 halts the buzzer 9 to sound the buzzer sound if the switch 6A and the switch 6C are simultaneously pushed. Namely, CPU 21 instructs the piezoelectric buzzer 9 to continue sounding until when a user recognizes that a measure against a heat disorder is noticed and pushes the switch 6A and the switch 6C to halt the buzzer sounding. Namely, such pushing two switches certainly makes a user further recognize noticing a preventive measure since the buzzer sounding is continued until when the user recognizes the noticed preventive measures. Here, a display 49 is displayed in the display region 46, which is shown in FIG. 9, during a period until the time when the display 48 is displayed. The display 49 is a display showing repetition of moving from left to right, allowing a user to recognize the operational mode of the heat indicator measuring device 1 as the preventive measure noticing mode. A display 50 as a timer display indicates elapsed time (or a period) since timing is started. A user recognizes a period until the time of supplying water by the display 50.

For example, if the temperature measured by the temperature sensor 2 is 27° C. and the relative humidity measured by the humidity sensor 3 is 50%, the WBGT approximated value is calculated as around 26° C. by the heat indicator calculation unit 5. CPU 21 determines that the WBGT level is “3” based on the calculated WBGT approximated value using the table (1). Here, the exercise level set in step S 30 is the “moderate exercise”. This exercise level allows the noticed contents in the table (1) at the time of the WBGT level “3” to become the two followings. Namely, in the contents, first, it takes 20 seconds to display a message of supplying water and sound a continuous monotonic buzzer sound along with the displaying and these displaying and sounding are performed every 20 minutes. Second, it takes 20 seconds to display a message of taking a rest and sound a short buzzer sound along with the displaying and these displaying and sounding are performed together every 30 minutes. Namely, as shown in FIG. 8(C), CPU 21 instructs the liquid crystal display 20 to display a display 51 (“DRINK”) as a message of supplying water for a user in the location of the displaying region 46 shown in FIG. 8 (A) and repeat it during 20 seconds every 20 minutes, and the piezoelectric buzzer 9 to sound a continuous monotonic buzzer sound during 20 seconds every 20 minutes along with the period and timing of displaying the display 51.

Further, as shown in FIG. 8(D), CPU 21 instructs the liquid crystal display 8 to display a display 52 (“REST-20”) as a message of taking a rest for a user in the location of the displaying region 46 shown in FIG. 8 (A) and repeat it during 20 seconds every 30 minutes, and the piezoelectric buzzer 9 to sound a short buzzer sound during 20 seconds along with the displaying of the display 52 every 30 minutes. Here, “20” in the display 52 indicates the period of taking a rest, namely 20 minutes. Accordingly, displaying a period of taking a rest promotes a user to take a rest during the displayed period. Hence, displaying the period of taking a rest can suggest a user to take a period which is necessary for taking a rest as a preventive measure against a heat disorder. Here, the display of the period for taking a rest is “20”. But, it may be changed to other appropriate period which is necessary for avoiding a heat disorder.

When a user looking at the display 51 and the display 52 displayed by the liquid crystal display 8, he/she recognizes that both supplying water and taking a rest in addition to it are necessary as preventive measures against a heat disorder. Further, the piezoelectric buzzer sounds a sound along with the display 52 and the display 52, preventing a user from missing out the instruction messages. Further, a user also recognizes the contents of preventive measures by hearing buzzer sounds since a sounding pattern of the buzzer sound is changed between the display 51 (“DRINK”) and the display 52 (“REST-20”). Here, the buzzer sounding is halted by pushing the switch 6A and the switch 6C simultaneously.

Here, if noticing supplying water every 20 minutes is concurrently performed with noticing taking a rest every 30 minutes, displaying a message indicating a taking a rest (“REST-20”) and the buzzer sounding (short sounding for 20 seconds) may be given preference over noticing supplying water. Otherwise, displaying a message indicating supplying water (“DRINK”) may be alternatively performed with displaying a message indicating a taking a rest (“REST-20”). Either a continuous short sound indicating the notice of supplying water or a short sound indicating the notice of taking a rest may be sounded by the buzzer.

For example, if the temperature measured by the temperature sensor 2 is 31° C. and the relative humidity measured by the humidity sensor 3 is 70%, the WBGT approximated value is calculated as around 34° C. by the heat indicator calculation unit 5. Then, CPU 21 determines that the WBGT level is “5” based on the calculated WBGT approximated value using the table (1). Here, the exercised level set in step S 30 is the “moderate exercise”. This exercise level allows the noticed content in the table (1) at the time of the WBGT level “5” to become a message of halting an exercise or a work and sound a continuous buzzer sound. As shown in FIG. 8(E), CPU 21 instructs the liquid crystal display 8 to display a display 53 (“DANGER”) as a message of halting an exercised or a work for a user in the display region 46 shown in FIG. 8(A). Further, it instructs the piezoelectric buzzer 9 to sound a continuous buzzer sound along with the display 53. A user can recognize the necessity of halting an exercise or a work as a preventive measure against a heat disorder by looking at the display 53 displayed by the liquid crystal display 8. Further, a buzzer sound sounded by the piezoelectric buzzer 9 along with the display 53 prevents a user from missing out a message. The display 53 as a message of halting an exercise or a work is displayed and a buzzer sound is sounded along with the displaying soon after the WBGT level as the WBGT approximated value is determined to be as “5”. Hence, a user can instantaneously acknowledge that doing an exercise or a work under such circumstance is dangerous. Here, making the buzzer sound at this time be larger than the buzzer sound instructing supplying water or taking a rest certainly notices a user about the current dangerous state. Displaying the display 53 and the continuous buzzer sound sounded by the piezoelectric buzzer 9 is continued until the time when the switch 6A and the switch 6C are simultaneously pushed.

Accordingly, in the heat indicator measuring device 1, a user can receive an appropriate preventive measure against a heat disorder corresponding to the measured WBGT approximated value, since he/she selects one of tables including noticed content data (the table (1) and the table (2)) corresponding to a user's physical strength in step S 20 and also selects any of exercise levels in step S 30 as being adjusted to an exercise level which a user is doing.

Namely, the degree of risks of suffering a heat disorder during an exercise heavily depends on a user's exercise level and a physical strength or weakness. The degree of risks of suffering a heat disorder during an exercise depending on a user's exercise level and a physical strength or weakness is fairly larger than the degree of risks of suffering a heat disorder in a daily life depending on individual characteristics such as ages and the like. Hence, in order to effectively avoid suffering a heat disorder during doing an exercise, it is necessary to take an appropriate preventive measure corresponding to a user's exercise level and a physical strength or weakness by certainly grasping a user's exercise level and his/her physical strength or weakness. Accordingly, holding data regarding a user's exercise level and his/her physical strength or weakness as user's data for determining a noticed content allows the noticed content, which indicates a preventive measure against a heat disorder during an exercise, to be optimized as a preventive measure against a heat disorder during an exercise.

Further, in the exemplary embodiment, the noticed contents include a specific instruction message for preventive measures against a heat disorder, a sound pattern of a buzzer sounding along with the instruction message, timing of displaying the instruction message and the sounding from the buzzer, and the length of a period for sounding of the buzzer sound and displaying of the instruction message. But, it may include only the displaying of an instruction message during a predetermined period every predetermined timing. The noticed contents in the exemplary embodiment are illustrated as examples. The details of instruction messages, timing for displaying instruction messages, and timing for sounding a buzzer sound are arbitrarily set in response to a user's physical strength or weakness, a WBGT level and an exercise level so as to optimally prevent a heat disorder.

The WBGT approximated value is calculated based on temperature and relative humidity averaged during a predetermined period such as two minutes. Namely, calculating the WBGT approximated value based on average temperature and average relative humidity during a predetermined period enables a user to receive a notice of an optimized preventive measure in response to the actual environment (temperature and relative humidity) around the user. On the other hand, in a case when a noticed content is determined based on the real time WBGT approximated value, if a direct sun light beam is instantaneously irradiated onto the detector 4 for example, the WBGT approximated value is incorrectly calculated under higher temperature circumstance although the actual temperature around a user does not become higher. Further, in a case when a user cooks food materials in boiling water in a well-ventilated room for example, a user stays in an environment having two areas; one area for cooking food materials in boiling water which has high relative humidity due to steams and the other area which does not have such relative high humidity. In such environment, if it takes little time for a user to cook materials in boiling water and most time to work in the other area, it is rare for the user to suffer a heat disorder since the period of doing job under high temperature (a period for a user to cook materials in boiling water) is short. However, if a noticed content is determined based on the real-time WBGT approximated value, the WBGT approximated value is calculated based on the relative humidity only during a short period of cooking materials in boiling water. As the result, an inappropriate preventive measure is noticed to a user such as case when halting an exercise and/or a sport is noticed although only the preventive measure of supplying water is sufficient under normal circumstance. However, as described above, calculating the WBGT approximated value based on averaged temperature and averaged relative humidity during a predetermined period can avoid the above issue. Here, the averaged temperature and averaged relative humidity may be obtained by averaging temperature and relative humidity after they are measured by a predetermined number every a predetermined interval.

If a user is changed or an exercise level is changed, the operational setting is changed by returning the operations to steps S 20 and S 30. Continuously pushing the switch 6D during 2 seconds enables the heat indicator measuring device 1 to return the initial state (step S 10) and its operation to be newly set. Here, if the switch 6D in the initial state (step S 10) is pushed under the initial state (step S 10), the operational mode of the heat indicator measuring device 1 is cyclically changed to the watch mode, the stop watch mode and the heat indicator measuring mode every pushing the switch 6D.

The contents such as WBGT levels, exercise levels and noticed contents are illustrated as examples in the tables (1) and (2). But, they may be arbitrarily changed. For example, a noticed content as a preventive measure against a heart disorder may be determined corresponding to WBGT levels and exercise levels, by referring to an operational guideline for a preventive measure against a heart disorder described in “a guidebook for preventive measures against a heart disorder during playing sports” published by the Japan sport association on Apr. 26, 1999 (revised on Jun. 3, 2006.)

The first exemplary embodiment of the heat indicator measuring device 1 was described above. As a modification of it, the heat indicator measuring device 1 may be provided with a biological object data measuring unit that measures biological object data such as user's body temperatures, pulses and blood pressures, and a table of noticed content data that specifies a noticed content viewed from three points such as a WBGT approximated value, an exercise level and biological data instead of the noticed content data in tables (1) and (2). This modification allows the noticed content as a preventive measure against a heat disorder to be formed based on user's biological data output from a biological sensor and sent to a user. If the heat indicator measuring device 1 is provided with a biological sensor, which is placed at the bottom of the heat indicator measuring device 1, namely at a position opposing to a front side where the detector 4 is placed, a biological sensor can be in close contact with a user's skin, certainly acquiring biological data from a user's body. Here, a user may input biological data by him/her self into the heat indicator measuring device 1 instead that a biological sensor measures biological data.

The heat indicator measuring device 1 is a wrist watch type mounted on a user's wrist in the first exemplary embodiment. But it may be a pendant type strung from a user's neck. Further, a noticing unit may include any one of the liquid crystal device 8 and the piezoelectric buzzer 9 instead of both of them. Further, a noticing unit may include a structure having an oscillation motor for noticing with oscillation, or sounding a voice for noticing instead of the piezoelectric buzzer 9.

As a memory unit memorizing a table of the noticed content data, an exchangeable memory such as a SD memory card may be used instead of ROM 20. In such case, the SD memory card memorizing a table of the noticed content data is prepared in advance, effectively and widely noticing preventive measures against a heat disorder in response to kinds of users and exercises. The noticed content data are kinds of users such as athletes, children and old persons, and kinds of exercises such as a work in a coal mine, a work in a bath room, running a marathon and playing a succor. Further, rewritable ROM may be used instead of ROM 20 so as to read a table of the noticed content data from other device such as a personal computer with wireless or wire means.

### Second Exemplary Embodiment

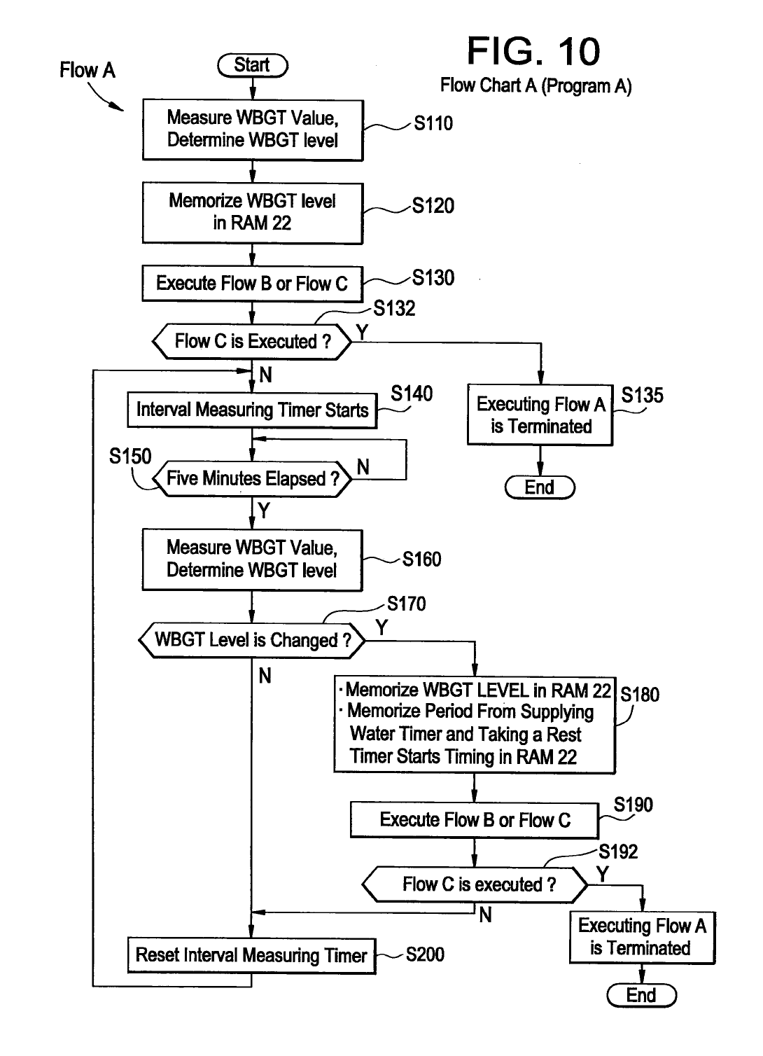

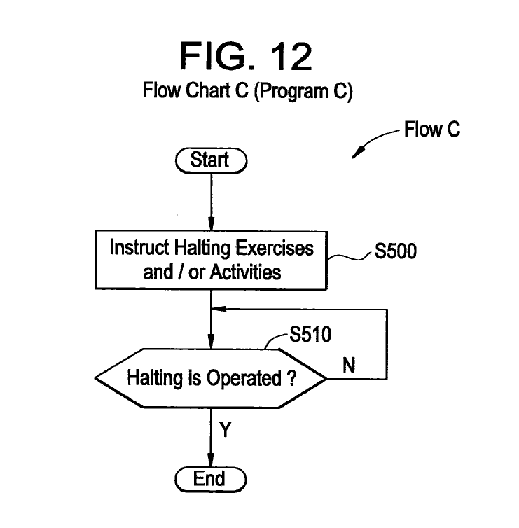

A work flow of a program for noticing preventive measures performed in the heat indicator measuring device 1 will be explained below as a second exemplary embodiment with referring to FIGS. 10 and 12. Using this program allows the heat indicator measuring device 1 to control the liquid crystal display device 8 and the piezoelectric buzzer 9 for displaying and sounding the noticed contents regulated in the tables (1) and (2). A method of controlling the heat indicator measuring device 1 will be explained along with the operations of the heat indicator measuring device 1.

A program for noticing a preventive measure is memorized by ROM 20. CPU 21 controls the operations of the liquid crystal display device 8 and the piezoelectric buzzer 9 by executing instructions of the program for noticing a preventive measure. Namely, CPU 21 works as a unit for controlling a noticing unit.

The structure of the heat indicator measuring device 1 in the second exemplary embodiment is the same structure of it in the first exemplary embodiment except that the program for noticing preventive measures is memorized in the ROM 20 and the operations of the liquid crystal display device 8 and the piezoelectric buzzer 9 are controlled by CPU 21 with executing the program for noticing preventive measures. Namely, as shown in the work flow of FIG. 5, the heat indicator measuring device 1 in the second exemplary embodiment executes the program for noticing preventive measures during the preventive measure noticing mode (step S50) based on WBGT levels determined by the selected table of noticed content data and the measured WBGT approximated value, after one of noticed content data tables (tables (1) and (2)) is selected in the table selecting mode (step S 20) and one of exercise levels is selected in the exercise level setting mode (step S 30.) In other words, CPU 21 controls the operations of the liquid crystal display device 8 and the piezoelectric buzzer 9 by executing the program for noticing preventive measures in the preventive measure noticing mode (step S50) after CPU acquires a kind of tables of the noticed content data selected in the table selecting mode (step S 20) and one of the exercise levels set in the exercise level setting mode (step S 30.) This control allows the liquid crystal display device 8 and the piezoelectric buzzer 9 to notice a user about preventive measures against a heat disorder.

The operations of the liquid crystal display 8 and the piezoelectric buzzer 9 by executing the program for noticing preventive measures will be explained with referring to FIGS. 10 to 12.

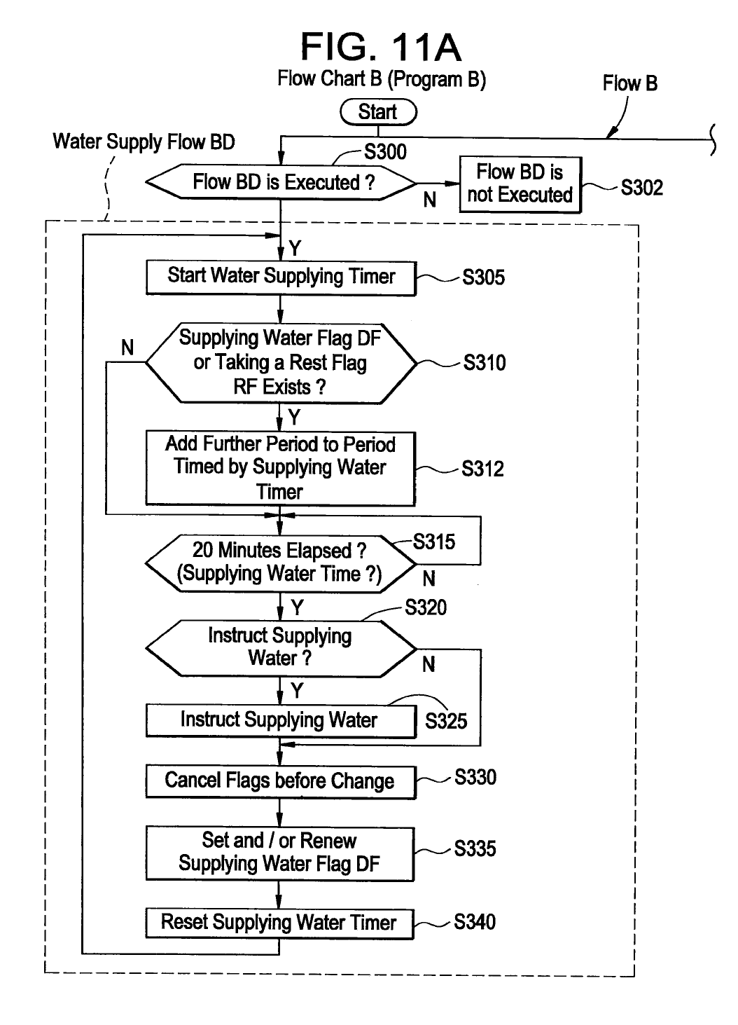

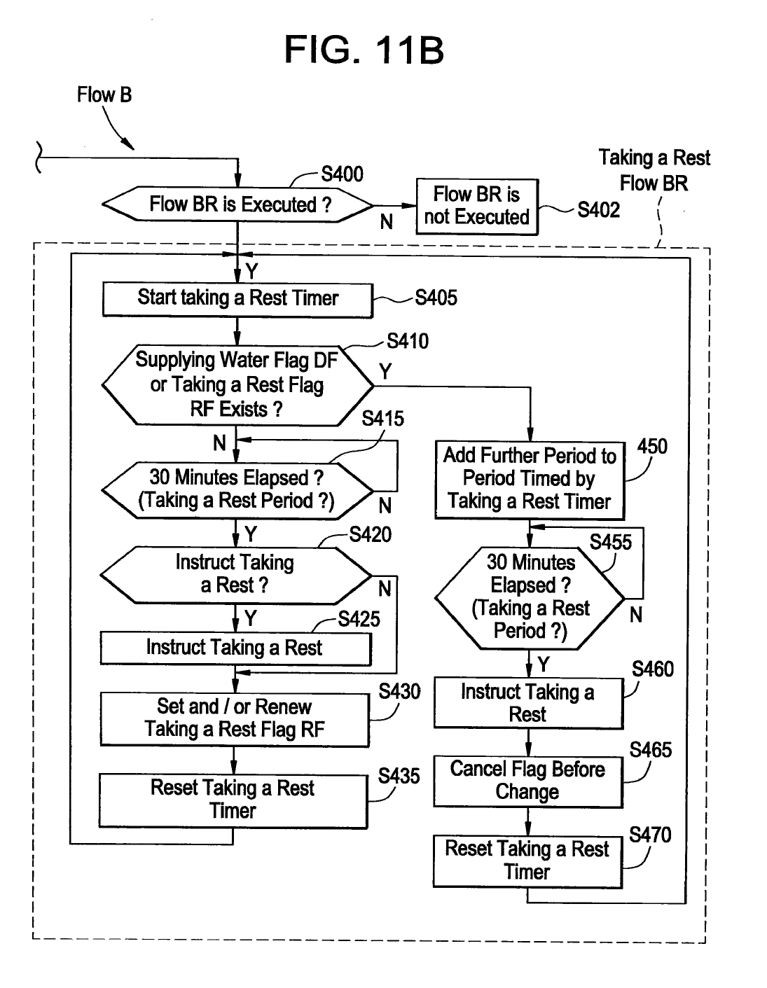

The program for noticing preventive measures comprises a program A for executing a flow chart A shown in FIG. 10, a program B for executing a flow chart B shown in FIG. 11, and a program C for executing a flow chart C shown in FIG. 12. In the preventive measure noticing mode (step S50), the program A for executing the flow chart A, the program B for executing the flow chart B, and the program C for executing the flow chart C are executed.

The processes indicated in the flow chart A (called as the flow A) is to control overall operations of the liquid crystal display 8 and the piezoelectric buzzer 9. On the other hand, the processes indicated in the flow chart B (called as the flow B) and the processes indicated in the flow chart C (called as the flow C) are to control specific operations of the liquid crystal display 8 and the piezoelectric buzzer 9.

Namely, the flow chart B is to control the operations of the liquid crystal display 8 and the piezoelectric buzzer 9 so that the liquid crystal display 8 and the piezoelectric buzzer 9 indicate a user about supplying water and taking a rest (see FIG. 11) in order that the user safely continues his/her exercises and/or exercises. The flow chart C is to control the operations of the liquid crystal display 8 and the piezoelectric buzzer 9 so as to indicate a user about halting his/her exercises and/or activities (see FIG. 12.)

First, the flow A will be explained referring to FIG. 10.

First, a temperature and humidity are measured and the WBGT approximated value is calculated based on the measured temperature and humidity. Then, CPU 21 determines a WBGT level based on the calculated WBGT approximated value (step S 110.) This WBGT level is memorized by RAM 22 (step S 120), and then the flow B shown in FIG. 11 described below or the flow C shown in FIG. 12 described below will be executed corresponding to a WBGT level (step S 130.) The relationship between a WBGT approximated value and a WBGT level is the same in tables (1) and (2) including noticed content data shown in FIGS. 5 and 6 respectively.

For example, in a case when table (1) including noticed content data is selected in the table selecting mode (step S 20), the intermediate level exercise is selected in the exercise level selection mode (step S 30) and the WBGT level is in the range from “1” to “3”, the execution of the flow B is started in step S 130. On the other hand, in a case when the table (1) including noticed content data is selected in the table selecting mode (step S 20), the intermediate level exercise is selected in the exercise level selection mode (step S 30) and the WBGT level is more than “4”, the execution of the flow C is started in step S 130.

If the flow B is executed (indicated as “No” in step S 132), the flow A is also simultaneously executed. Namely, steps from S 140 to S 200 in the flow A described below are executed during the execution of the flow B. On the other hand, if the flow C is executed (indicated as “Yes” in step S 132), the execution of the flow A is terminated (step S 135.)

In the flow C, as shown in FIG. 12, a risk of continuing exercises and/or activities is noticed to a user and halting exercises and/or activities is instructed to a the user (step S500.) More particularly, as shown in FIG. 8, the display 53 (“DANGER”) is displayed by the liquid crystal display 8 and a continuous buzzer sound is sounded by the piezoelectric buzzer 9. Then, it is determined whether both the switches 6A and 6C are pushed together and displaying the display “53” and sounding a buzzer sound by the piezoelectric buzzer 9 are halted or not (step S510.) The display 53 (“DANGER”) displayed by the liquid crystal display 8 and a continuous buzzer sound sounded by the piezoelectric buzzer 9 are continued until these pushing and halting are performed (indicated as “No” in step S510.) If the halting operation is executed (indicated as “Yes” in step S510), the execution for indicating the halt of exercises and/or activities is ended (S500) and the flow C is terminated.

The flow C includes processes for making a user halt his/her exercises and/or activities. Accordingly, it is necessary not to instruct a user about supplying water and taking a rest on the premise of the continuation of exercises and/or activities soon after the flow C is executed. Hence, the flow A is terminated in a case when the flow C is executed (step S 135.)

After the flow C is terminated (indicated as “Yes” in step S510) by pushing both the switches 6A and 6C and halting the displaying of the display 53 and the sounding of the continuous sound by the piezoelectric buzzer 9, there is a case when a user would like to start his/her exercises and/or activities again under a circumstance having an appropriate temperature and humidity for his/her exercises and/or activities after a while. In such case, if the switch 6D is pushed and the operational mode of the heat indicator measuring mode 1 is moved to the heat indicator measuring mode and then a predetermined operation is set during the operation setting mode (from step S 10 to step S 40), the preventive measure noticing mode (step S50) can be executed.

On the other hand, when the flow B is executed in step S 130, a interval measuring timer for controlling an interval of measuring WBGT approximated values starts timing in the flow A. This interval measuring timer is functioned by the CPU 21.

The interval measuring timer times five minutes for example (step S 150.) If the interval measuring timer times five minutes (indicated as “Yes” in step S 150), a WBGT approximated value is calculated again and a WBGT level is determined based on the calculated WBGT approximated value (step S 160.) Then, it is determined whether the determined WBGT level is changed from the WBGT level memorized in RAM 22 (step S 170.) CPU 21 executes this determination of the change of the WBGT level. Namely, CPU 21 works as a unit for determining the change of a heat indicator.

In the exemplary embodiment, CPU 21 determines the predetermined change of the heat indicator as the change of WBGT levels regulated in tables (1) and (2) including noticed content data. Here, the WBGT levels are not limited to the contents regulated in tables (1) and (2), but the range of the heat indicator (numerical values) corresponding to each level may be narrowed or widened. Otherwise, if the WBGT level is changed as equal to or more than 2 levels, it may be determined that the WBGT level be changed as a predetermined change. In the exemplary embodiment, CPU 21 determines the change of the WBGT level every five minute interval. But, this interval is not limited to this value, but may be shorter or longer. Shortening the interval effectively prevents against a heat disorder corresponding to the change of a WBGT level.

If it is determined that the WBGT level is changed in the step S 170 (indicated as “Yes” in step S 170), the WBGT level previously memorized in step S 120 is replaced with the WBGT level after the change and the changed WBGT level is memorized in RAM 22 (step S 180.) Further, in step S 180, a period after a supplying water timer in the flow B (step S 305) and a taking a rest timer (step S 405) described later starts timing is also memorized. Here the period after a supplying water timer in the flow B (step S 305) and a taking a rest timer (step S 405) described later starts timing is a thing in the flow B which is already executed. Next, one of the flow C and the flow B is selected depending on the changed WBGT level. If the flow B is selected in the step S 190, the flow B which has been already executed is terminated. Then, the new flow B based the changed WBGT level is executed.

The interval measuring timer is reset (indicated as “No” in step S 170) after the execution of the flow B is started (step S 190) or when it is determined that the WBGT level is not changed at step S 170 (indicated as “Yes” in step S 170). Then, timing by the interval measuring timer is started again (step S 140) and the executions of steps from S 150 to S 200 are repeated thereafter. Namely, the flow A is executed concurrently with execution of the flow B in step S 130 or step S 190. On the other hand, when the flow C is selected at the step S 190, the flow B on running is terminated and the flow C is executed. When the flow C is executed (indicate as “Yes” in step S 192), the flow A is terminated not so as to indicate a user about supplying water and taking a rest on the premise of continuation of exercises and/or activities (step S 195.)

As described above, the flow A is executed concurrently with the execution of the flow B in the preventive measure noticing mode (step S50), measuring the WBGT approximated value every a predetermined interval (every 5 minutes in the exemplary embodiment) and determining the WBGT level. Then, if the WBGT level is changed, the flow B is executed along the content corresponding to the changed WBGT level. If the changed WBGT level is a halt of user's exercises and/or activities, executing the flow C prevents the user from suffering from a heat disorder.

Next, executions of the flow B executed in steps 130 and S 190 will be explained referring to the flow chart shown in FIG. 11. The flow B comprises a supplying water flow BD that indicates a user about supplying water and a taking a rest flow BR that indicates a user about taking a rest.

In the flow B, first, it is determined whether the executions of a supplying water flow BD is started or not (step S 300) and it is also determined whether the executions of a taking a rest flow BR is started or not (step S 400), based on the table of noticed content data (the table (1) or (2)) selected in the table selecting mode (step S 20), an exercise level set in the exercise level setting mode (step S 30) and the WBGT level measured and determined in steps S 110 or S 160.

Here, a case where the table (1) is selected in the table selecting mode (step S 20), the intermediate exercise level is set in the exercise level setting mode (step S 30) and the WBGT level is determined as “2” in steps S 110 or S 160 will be explained. In this case, as shown in the table (1) including noticed content data, only supplying water is instructed to a user. Namely, “DRINK” is displayed by the liquid crystal display 8 and a continuous and short buzzer sound is sounded by the piezoelectric buzzer 9 during 20 seconds every 20 minutes. Taking a rest is not instructed to a user. Namely, starting the supplying water flow BD is determined in step S 300 of the flow B (indicated as “Yes” in step S 300). Further, no execution of the taking a rest flow BR is determined in step S 400 of the flow B (indicated as “No” in step S 400) and the execution of halting the taking a rest flow BR is maintained (step S 403.

In the supplying water flow BD, a supplying water timer starts timekeeping so as to time the timing of instructing supplying water after the determination of starting the executions of the supplying water flow BD (indicated as “Yes” in step S 305.) This supplying water timer is functioned by the CPU 21.

Next, it is determined whether one of a supplying water flag DF and a taking rest flag RF exists or not (step S 310.) The supplying water flag DF and the taking rest flag RF are set in the supplying water flow BD and the taking a rest flow BR respectively that have been executed before the change of the WBGT level described later. Here, first, a case where the supplying water flag DF and the taking rest flag RF are not set before the change of the WBGT level will be explained. “No” is determined at step S 310 in this case. Then, it is determined whether 20 minutes as the predetermined interval elapsed or not (step S 315) from the time when timing by the supplying water timer is started (step S 305) or not and it is also simultaneously determined whether supplying water is instructed to a user or not (step S 320.)

When it is determined that 20 minutes elapsed (indicated as Yes in step S 315) since timing by the supplying water timer is started (step S 305) and it is also determined that supplying water is instructed to a user ((indicated as “Yes” in step S 320), “DRINK” is displayed by the liquid crystal display 8 and a continuous short buzzer sound is sounded by the piezoelectric buzzer during 20 seconds (step S 325.)

On the other hand, if the instruction of taking a rest in the taking a rest flow BR (steps S 425 and S 460) described later is overlapped with the instruction of supplying water (step S 325), the instruction of taking a rest is prioritized and supplying water is not instructed even in a case when “No” was determined in step S 320 and 20 minutes elapsed after a supplying water timer has started timing (step S 305.)

Next, setting the supplying water flag DF and the taking a rest flag RF before the change of the WBGT level, which was the object to be determined in step S 310, is cancelled (step S 330), after supplying water is instructed to a user (step S 325), or non instruction of supplying water is determined in step S 320 (indicated as “No” in step S 320.) But cancellation of setting the flags in step S 330 is not performed since it is determined that there is no existence of the supplying water flag DF before the change of the WBGT level (flag before change) and no existence of the taking a rest flag RF (flag before change).

Next, the supplying water flag DF is set as a flag indicating that supplying water was instructed to a user in step S 325 (step S 335.) Here, the supplying water flag DF is set (step S 335) even if it is determined that supplying water is not instructed in step S 320 (indicated as “No” in step S 320.) Then, the supplying water timer is reset (step S 340) and the execution is back to step S 305. Further, in addition to the display “DRINK” displayed by the liquid crystal display 8 and a continuous short buzzer sound sounded by the piezoelectric buzzer 9 every 20 minutes, renewing the supplying water flag DF set in advance is repeated (from step S 310 to S 330.) Here, the supplying water flag DF is renewed even in a case when it is determined that supplying water is not instructed (indicated as “No” in step S 320.)

Accordingly, the supplying water is instructed as a preventive measure against a heat disorder every 20 minutes, avoiding a heat disorder. Here, 20 minutes interval for instructing the supplying water is a predetermined interval for effectively preventing a heat disorder. This interval is not limited to 20 minutes, but arbitrarily set corresponding to user's data contents.

When the supplying water flag DF is set (step S 335), the WBGT level under the circumstance where the supplying water flow BD is executed is given to the supplying water flag DF as an identifier. Namely, the WBGT level determined in steps S 110 and S 160 is given to the supplying water flag DF as an identifier. Hence, in step S 310, identifying an identifier regarding the WBGT level given to the supplying water flag DF can determine the detail content of the supplying water flag DF. Namely, it can be determined whether the supplying water flag DF is one regarding the supplying water flow BD which is currently executed, or the other set in the supplying water flow BD or the taking a rest flow BR which has been executed before the change of the WBGT level.

Here, processes in a case when it is determined that the supplying water flag DF or the taking a rest flag RF before the change of the WBGT level exists in step S310 (indicated as “Yes” in step S 310) will be explained later.

Next, a case where the table (1) including noticed content data is selected in the table selecting mode (step S 20), the intermediate exercise level is set in the exercise level setting mode (step S 30) and the WBGT level is determined as “3” in steps S 110 or S 160, will be explained as example. In this case, as shown in the table (1), supplying water and taking a rest are instructed to a user. Namely, “DRINK” is displayed by the liquid crystal display 8 and a continuous and short buzzer sound is sounded by the piezoelectric buzzer during 20 seconds every 20 minutes.

The instruction of taking a rest is that the liquid crystal display 8 displays “REST-20” and the piezoelectric buzzer sounds a short buzzer sound during 20 seconds every 30 minutes. The instruction of supplying water is executed by performing the supplying water flow BD described above and followed by determining that the execution of the supplying water flow BD is started in step S 300 (indicated as “Yes” in step S 300.) Further, the instruction of taking a rest is executed by performing executions after step S 405 described below and followed by determining that the execution of the taking a rest flow BR is started in step S 400 (indicated as “Yes” in step S 400.)

In the taking a rest flow BR, the taking a rest timer starts timing so as to time the timing of instructing taking a rest after the determination of starting the executions of the taking rest flow BR (indicated as “Yes” in step S405.) This taking a rest timer is functioned by the CPU 21.

Next, it is determined whether the supplying water flag DF and the taking a rest flag RF exist or not (step S410.) The supplying water flag DF and the taking rest flag RF are set in the supplying water flow BD and the taking a rest flow BR respectively that have been executed before the change of the WBGT level described later. Here, First, a case where the supplying water flag DF and the taking rest flag RF are not set before the change of the WBGT level will be explained. “No” is determined at step S410 in this case. Then, it is determined whether 30 minutes elapsed from the time when the taking a rest timer (step S405) starts timing or not and concurrently determined whether taking a rest is instructed to a user or not (step S420.)

When it is determined that 30 minutes elapsed (indicated as “Yes” in step S415) from the time when the taking a rest timer starts timing (step S 405) and concurrently determined that taking a rest is instructed to a user ((indicated as Yes in step S420), “REST-20” is displayed by the liquid crystal display 8 and a continuous short buzzer sound is sounded by the piezoelectric buzzer during 20 seconds (step S 425.)

On the other hand, if timing of taking a rest is already determined such as a half time during a game and this timing is overlapped with the timing of taking a rest in step S 425 for example, “No” is determined in step S 420 and taking a rest is not instructed to a user even when 30 minutes elapsed after the taking rest timer starts timing (step S 405.) Here, timing of a half time is memorized in advance in the operation setting mode (any one of stages from step S 10 to step S 40) by RAM 22 or the like.

Subsequently, a flag of instructing a user about taking a rest is set as the taking a rest flag RF (step S 430) after the taking a rest is instructed to a user (step S 425) or it is determined that taking a rest was not instructed to a user (indicated as “No” in step S 420.) Here, the taking a rest flag RF is set (step S430) even if it is determined that taking a rest is not instructed in step S420 (indicated as “No” in step S420.)

Then, the taking a rest timer is reset (step S 435) and the process is back to step S 405. In addition to the display “REST-20” displayed by the liquid crystal display 8 and a continuous short buzzer sound sounded by the piezoelectric buzzer 9, renewing the taking a rest flag RF set in advance is repeated (from step S 410 to S 430.) Here, the taking a rest flag RF is renewed even in a case when it is determined that taking a rest is not instructed (indicated as “No” in step S 420.)

Accordingly, the taking a rest is instructed as a preventive measure against a heat disorder every 30 minutes, avoiding a heat disorder. Here, 30 minutes interval for instructing the taking a rest is a predetermined interval for effectively preventing a heat disorder. This interval is not limited to 30 minutes, but arbitrarily set corresponding to user's data contents.

When the taking a rest flag RF is set (step S 430), the WBGT level under the circumstance where the taking a rest flow BR is executed is given to the taking a rest flag RF as an identifier. Namely, the WBGT level determined in steps S 110 and S 160 is given to the taking a rest flag RF as a identifier. Hence, in step S 410, identifying an identifier regarding the WBGT level given to the taking a rest flag RF can determine the detail content of the taking a rest flag RF. Namely, it can be determined whether the taking a rest flag RF is one regarding the taking a rest flow BR which is currently executed, or the other set in the supplying water flow BD or the taking a rest flow BR which has been executed before the change of the WBGT level.

Here, executions in a case when it is determined that the supplying water flag DF or the taking a rest flag RF before the change of the WBGT level exists in step S410 (indicated as “Yes” in step S 410) will be explained later.

As described before, in the flow A, the existence or non existence of the change of the WBGT is determined every 5 minutes and the execution of the flow B or the flow C is selected corresponding to the changed WBGT level (step S190.) If the flow B is selected, the flow B which has been already executed is terminated. Then, the new flow B based the changed WBGT level is executed. Namely, when the executions of the supplying water flow BD and the taking a rest flow BR in the flow B are started, the supplying water timer and the taking a rest timer start timing at the time of starting these executions. Hence, this starting timing allows the interval for instructing the supplying water and the interval for instructing the taking a rest to be timed from the beginning.

Hence, if the WGBT level is changed from “3” to “2”, there is case where the instruction of a preventive measure against a heat disorder which has been performed at end before this change is an instruction of supplying water which has been performed ten minutes before the change. However, the instruction of supplying water should be performed every 20 minutes in the flow B. Hence, supplying water is instructed after 20 minutes from the time when the timing of step S 305 in the flow B is started after the WGBT level is changed from “3” to “2”. Namely this execution causes the instruction of supplying water not to be performed during 30 minutes from the previous instruction of supplying water, lacking an appropriate measure against a heat disorder.

Hence, in order to avoid such inappropriateness, it is determined in step S 310 and step S 410 whether the supplying water flag DF or the taking a rest flag RF set in the flow B which has been executed before the change of WBGT level exists or not. If it is determined that any one of these flags exists (indicated as “Yes” in step S 310 or “Yes” in step S 410) a predetermined execution (step S 312 or step S 450) is performed.

More specifically, when the WBGT level is changed from “3” to “2” for example, the supplying water flag DF is set in step S 335 and the taking a rest flag is set in step 430 in the flow B which has been executed when the WBGT level before change was “3”. Hence, it is determined that one of the supplying water flag DF and the taking a rest flag RF before the change of WBGT level exists in step S 310. This step is the supplying water flow BD of the flow B which is executed after the WBGT level is changed from “3” to “2” (indicated as “Yes” in step S 310.)

Further, in this case (indicated as “Yes” in step S 310), a further period is added to a period timed by the supplying water timer in step S 305 (step S 312.) The further period is a period after the preventive measure against a heat disorder is instructed at end before the change of the WBGT level until the time when the supplying water timer starts timing in the flow B which is executed after the change of the WBGT level. This addition allows the first instruction of supplying water after the change of WBGT level from “3” to “2” to be performed 20 minutes later as the predetermined period from the time when the instruction of supplying water or taking a rest has been performed before the change of the WBGT level. Namely, missing the timing for supplying water as a preventive measure against a heat disorder can be avoided by these executions. 20 minutes as this predetermined period is the same of the interval set in S 315 and this setting can effectively prevent a heat disorder. Here, the predetermined period may be shorter or longer than the predetermined interval set in S 315 if it effectively prevents a heat disorder.

Here, a period which is shorter among periods from the time when the supplying water timer and the taking a rest timer starts timing in the flow B executed before the change of the WBGT level, which are memorized in step S 180 of the flow A, is set to be a period from the time when the instruction of the preventive measure is performed at end before the change of the WBGT level to the time when the supplying water timer starts timing in the flow B executed after the change of the WBGT level.

Further, for example, when the WBGT level is changed from “2” to “3”, the supplying water flag DF is set in step S 335 of the flow B executed when the WBGT level before the change was “2”. Accordingly, in step 310 of the supplying water flow BD and step S 410 of the taking a rest flow BD in the flow B executed after the WBGT level is changed from “2” to “3”, it is determined that the supplying water flag DF set before the change of the WBGT level exists (indicated as “Yes” in step S 310 and “Yes” in step S 410.)

In this case, the execution which is the same in the case when the WBGT level is changed from “3” to “2” describe above is performed in the supplying water flow BD. The first instruction of supplying water after the WBGT level is changed from “2” to “3” is performed 20 minutes later from the time of the instruction of the supplying water or the taking a rest executed before the change of the WBGT level.

On the other hand, in the taking a rest flow BR, a further period from the time after the preventive measure against a heat disorder is instructed at end before the change of the WBGT level until the time when the taking a rest timer starts timing in the flow B executed after the change of the WBGT level is added to a period timed by the taking a rest timer in step S 405 (step S 450). Then, the taking a rest timer determines whether it times 30 minutes including the added period or not (step S 455). If 30 minutes are timed by a timer (indicated as “Yes” in step S 455), the display “REST-20” is displayed by the liquid crystal display 8 and a short sound is sounded by the piezoelectric buzzer 9 during 20 seconds (step S 460.) This execution allows the first instruction of supplying water after the change of WBGT from “2” to “3” to be performed 30 minutes later as a predetermined period from the instruction of supplying water or taking a rest executed before the change of the WBGT level. Namely, missing timing of the taking rest as a preventive measure against a heat disorder can be avoided. Here, when taking a rest, a user generally takes water. Hence, supplying water is also actually instructed, effectively preventing a heat disorder. 30 minutes as this predetermined period is the same of the predetermined interval set in S 415 and this setting can effectively prevent a heat disorder. Here, the predetermined period may be shorter or longer than the predetermined interval set in S 415 if it effectively prevents a heat disorder.

Here, similarly to the taking a rest flow BR, a period which is shorter among periods from the time when the supplying water timer and the taking a rest timer start timing in the flow B executed before the change of the WBGT level, which are memorized in step S 180 of the flow A, is set to be a period from the time when the instruction of the preventive measure is performed at end before the change of the WBGT level to the time when the taking a rest timer starts timing in the flow B executed after the change of the WBGT level.

Then, setting the supplying water flag DF before the change of the WBGT level is cancelled (step S 465) and the taking a rest timer starts timekeeping again (step S 405) after it is reset (step S 470.) In step S 410, it is determined that the water flow flag DF does not exist (indicated as “No” in step S 410) since setting the supplying water flag DF before the change of the WBGT level is cancelled in step S 465. Further, the executions from step S 410 to step S 440 are repeated and the instruction of taking a rest is displayed by the liquid crystal display 98 and is sounded by the piezoelectric buzzer 9 every 30 minutes.

In the second exemplary embodiment, two cases of the changes of the WBGT levels from “2” to “3” and from “3” to “2” were explained. But, even in other cases when the WBGT levels are changed from “1” to “2” and from “2” to “1,” a preventive measure against a heat disorder after the change of the WBGT level is similarly instructed with appropriate timing, based on the supplying water flag DF and the taking a rest flag RF before the change of the WBGT level.

In the taking a rest flow BR in the heat indicator measuring device 1 in the second exemplary embodiment, it is determined whether any of the supplying water flag DF and the taking a rest flag RF exists or not in step S 410. This determination avoids an extremely longer period until taking a rest is instructed. However, it may be determined only the existence of taking a rest flag RF in step S 410. In such case, the addition of the further period to a period timed by the taking a rest timer in step S 450 is performed only in a case when taking a rest is instructed before the change of the WBGT level. Namely, a further period after the time when taking a rest is instructed at end before the change of the WBGT level until the time when the taking a rest timer starts timing in the taking flow BR executed after the change of the WBGT level is added to a period timed by the taking a rest timer in step S 405. Even in this case, supplying water is instructed the predetermined period later (20 minutes) in the supplying water flow BD from the instruction of supplying water executed before the change of the WBGT level. Namely this instruction appropriately performs a preventive measure against a heat disorder.

Further, the program B of the second exemplary embodiment regarding the heat indicator measuring device 1 handles one case when only the instruction of the supplying water is executed (the supplying water flow BD) and the other case when the execution for instructing the supplying water ((the supplying water flow BD) is combined with the execution for instructing the taking a rest (the taking a ret flow BR). But, only the instruction of the taking a rest may be executed corresponding to a WBGT level. In this case, it is determined that the supplying water flow BD is not executed (indicated as “No” in step S 300) at the time when it is determined whether the execution of the supplying water flow BD is started or not in step S 300 of the flow B and the execution of the supplying water flow BD is held to be halted (step S 302.) On the other hand, in step S 400 of the flow B, it is determined that the execution of the taking a rest flow BR is started (indicated as “Yes” in step S 400) and only the taking a rest flow BR is executed, instructing only taking a rest.

Further, timing of supplying water and taking a rest is not limited to the interval of 20 minutes or 30 minutes described above. An arbitrary predetermined interval for effectively preventing a heat disorder can be set in taking consideration of kinds of users such as physical strength or weakness, sexes and ages, exercise and/or activity levels and a place for exercising.

The preventive measure noticing program is memorized by ROM 20 described above. Otherwise, an exchangeable memory card such as a SD memory card may memorize the preventive measure noticing program corresponding to kinds of users such as athletes, children and old persons, and kinds of exercises such as a work in a coal mine, a work in a bath room, running a marathon and playing a succor. Such SD card may be exchanged corresponding to kinds of users, places for exercising and the contents of them. Such memorization enables the device to widely and appropriately notice preventive measures against heat disorder. Further, rewritable ROM may be used instead of ROM 20 so as to read the tables of the noticed content data from other device such as a personal computer with wireless or wire means.

Further, in the second exemplary embodiment regarding the heat indicator measuring device 1, the program instructing a user about preventive measures against heat disorder includes the programs B and C. But, other programs such as changing interval of supplying water and taking a rest and instructing supplying minerals and/or cooling a head may be provided and an appropriate program among these programs may be executed corresponding to the user data contents and heat indicators.

In the exemplary embodiments regarding the heat indicator measuring device 1 described above, a biological object is a human being. But, it may be a pet such as a dog, a cat and domestic animals such as a horse, a pig, a cattle and the like. In the case when the exemplary embodiments are applied to a pet and a domestic animal as a biological object, a table of noticed content data which is suitable for each of objects is used.

1. A heat indicator measuring device comprising:
a temperature measuring unit that measures a temperature;
a humidity measuring unit that measures humidity;
a heat indicator calculation unit that calculates a heat indicator from the measured temperature and humidity;
a user data inputting unit that inputs user data as data regarding a user;
a noticed content memorizing unit that memorizes a noticed content including a content to be noticed and indicating a preventive measure against a heat disorder corresponding to the heat indicator and the user data;
a noticed content determining unit that determines the noticed content based on the heat indicator and the user data;
a noticing unit that notices the determined noticed content; and
a supporting unit supporting these units.

2. The heat indicator measuring device according to claim 1, wherein the noticed content includes an instruction message, a pattern of a sound sounded by a buzzer along with the instruction message, and timing of a display of the instruction message and sounding sounded by the buzzer.

3. The heat indicator measuring device according to claim 1, wherein the noticed content memorizing unit is exchangeable.

4. The heat indicator measuring device according to claim 1, wherein the heat indicator calculation unit calculates an average of temperatures measured during a predetermined period or by a predetermined number and uses the averaged temperature to calculate the heat indicator.

5. The heat indicator measuring device according to claim 1, wherein the heat indicator calculation unit calculates an average of humidity measured during a predetermined period or by a predetermined number and uses the averaged humidity to calculate the heat indicator.

6. The heat indicator measuring device according to claim 1, further comprising a biological object data measuring unit that measures the biological data of the user, wherein the noticed content memorizing unit memorizes a noticed content corresponding to the measured biological object data in addition to the heat indicator and the biological object data, and the a noticed content determining unit determines the noticed content based on the biological object data and the heat indicator and the measured biological object data.

7. The heat indicator measuring device according to claim 1, further comprising a wearing unit that is used for wearing the device with a body of the user, wherein a detector of the temperature measuring unit and the humidity measuring unit is placed on a position opposing a side of the body when the device is mounted on the body of the biological object by the wearing unit.

8. The heat indicator measuring device according to claim 7, wherein the biological object data measuring unit is placed in a position opposing to the body of the user.

9. A heat indicator measuring device comprising:
a temperature measuring unit that measures a temperature;
a humidity measuring unit that measures humidity;
a heat indicator calculation unit that calculates a heat indicator from the measured temperature and humidity;
a user data inputting unit that inputs user data as data regarding a user;
a noticing unit that notices the user about a preventive measure against a heat disorder;
a memory unit that memorizes a program for controlling the noticing unit to notice the preventive measure based on the heat indicator and the user's data;
a noticing unit controlling unit that controls the noticing unit based on the program; and
a supporting unit supporting these units.

10. The heat indicator measuring device according to claim 9, wherein the program makes the noticing unit to notice any one of supplying water and taking a rest or both of them as the preventive measure against heat disorder.

11. The heat indicator measuring device according to claim 9, wherein the program controls the noticing unit to notice any one of noticing supplying water and taking a rest or both of them every each of predetermined intervals.

12. The heat indicator measuring device according to claim 9, further comprising a heat indicator change determining unit that determines a change of the heat indicator along elapse of time and a condition of the change, wherein if the heat indicator change determining unit determines that there is a predetermined change of the heat indicator, the program controls the noticing unit based on the user data and the heat indicator after the determination of the predetermined change.

13. The heat indicator measuring device according to claim 9, wherein if the heat indicator change determining unit determines that there is a predetermined change of the heat indicator, the program controls the noticing unit to make a first notice after the determination at the time when a predetermined period elapses from the time of previous noticing before it is determined that there is the predetermined change.

14. The heat indicator measuring device according to claim 13, wherein the predetermined period is the predetermined interval.

15. A method of controlling a heat indicator measuring device comprising:
measuring an atmospheric temperature;
measuring humidity;
calculating a heat indicator from the measured temperature and humidity;
obtaining user data as data regarding a user; and
controlling a noticing unit to notice a preventive measure against a heat disorder based on the heat indicator and the user data.
